(19) United States
(12) Patent Application Publication
(10) Pub. No.: US 2025/0169726 A1
(43) Pub. Date: May 29, 2025

(54) Interferometric Near Infrared Spectroscopy System and Method

(71) Applicant: CoMind Technologies Limited, London (GB)

(72) Inventor: William PARKER, London (GB)

(21) Appl. No.: 18/869,048

(22) PCT Filed: May 26, 2023
(86) PCT No.: PCT/GB2023/051396

§ 371 Date: Nov. 25, 2024

(30) Foreign Application Priority Data
May 26, 2022 (GB) 2207777.0

(51) Int. Cl. A61B 5/1455; A61B 5/026; G01N 21/359; G01N 33/49

(52) U.S. Cl. CPC A61B 5/14552; A61B 5/0261; A61B 5/14553; G01N 21/359; G01N 33/4925; A61B 2562/0233; G01N 2201/068

(57) ABSTRACT

An aspect of the disclosure provides an interferometric near infrared spectroscopy, iNIRS, system comprising: a light emitting arrangement comprising: a first light source configured to provide wavelength-swept emission of light through a plurality of different wavelengths in a first wavelength range; a second light source configured to provide wavelength-swept emission of light through a plurality of different wavelengths in a second wavelength range different to the first wavelength range; a common sample delivery channel coupled to each of the first and second light sources to receive light therefrom and to deliver said light towards a subject; and one or more reference channels for receiving reference light from the first light source and/or the second light source; a light detecting arrangement comprising an interferometrie optical detector; wherein the optical detector is coupled to the one or more reference channels for receiving reference light from the first and second light sources; wherein the light detecting arrangement is arranged to be coupled to the subject for the optical detector to receive sample light from the first and second light sources, the sample light comprising light which travelled along the common sample delivery channel; wherein the optical detector is arranged to combine the sample light with the reference light to provide combined light signals comprising one or more components at a beat frequency between sample light and reference light; and wherein the iNIRS system is configured to process the combined light signals for providing imaging and analysis of the subject.

### TECHNICAL FIELD

The present disclosure relates to the field of imaging and analysis. In particular, the present disclosure relates to interferometric near infrared spectroscopy (‘iNIRS’) systems and methods for neuroimaging and analysis.

### BACKGROUND

Near infrared spectroscopy (‘NIRS’) is a spectroscopic method which uses the near infrared region of the electromagnetic spectrum (e.g. between 780 and 2500 nm). NIRS systems can be used to provide non-invasive monitoring of scattering and absorption properties of a medium. Radiation at NIRS wavelengths is less easily absorbed by human skin (and also bones) than visible light, and so NIRS radiation may penetrate both skin and skull, and penetrate into brain tissue. NIRS may be used as a technique for non-invasive imaging of human brain tissue by monitoring scattering and absorption properties of the NIRS radiation within the brain tissue.

It is desirable to provide improved technology for imaging and analysis.

### SUMMARY

Aspects of the disclosure are set out in the independent claims and optional features are set out in the dependent claims. Aspects of the disclosure may be provided in conjunction with each other, and features of one aspect may be applied to other aspects.

In an aspect, there is provided an interferometric near infrared spectroscopy, iNIRS, system (e.g. for neuroimaging and analysis) comprising: a light emitting arrangement comprising: a first light source configured to provide wavelength-swept emission of light through a plurality of different wavelengths in a first wavelength range (e.g. above an oximetry isosbestic wavelength); a second light source configured to provide wavelength-swept emission of light through a plurality of different wavelengths in a second wavelength range different to the first wavelength range (e.g. below the oximetry isosbestic wavelength); a common sample delivery channel coupled to each of the first and second light sources to receive light therefrom and to deliver said light towards a subject (e.g. towards their scalp); and one or more reference channels for receiving reference light from the first light source and/or the second light source; a light detecting arrangement comprising an interferometric optical detector. The optical detector is coupled to the one or more reference channels for receiving reference light from the first and second light sources. The light detecting arrangement is arranged to be coupled to the subject (e.g. to their scalp) for the optical detector to receive sample light from the first and second light sources, the sample light comprising light which travelled along the common sample delivery channel (e.g. which was delivered to the subject, such as to their brain tissue). The optical detector is arranged to combine the sample light with the reference light to provide combined light signals comprising one or more components at a beat frequency between sample light and reference light. The iNIRS system is configured to process the combined light signals for providing imaging and analysis (e.g. of the subject's brain).

Embodiments may enable a single light emitting arrangement to provide light emission in two separate wavelength ranges, where light from each light source is emitted via at least one common optical channel (e.g. where light from both light sources is emitted along the same optical channel). Embodiments may enable a single light detecting arrangement to receive both sample and reference light from the two separate light sources, via one or more common optical channels. Use of light in wavelength ranges either side of the oximetry isosbestic wavelength may enable blood oxygen concentrations to be determined for the subject's brain tissue.

Each light source may be arranged to emit a series of pulses, each pulse comprising at least one sweep through the plurality of different wavelengths. Each light source may be configured to sweep linearly through the plurality of different wavelengths. Light from each light source may travel along at least one common optical path, e.g. a single optical channel, as it travels from the light source to the subject's scalp. In other words, the common sample delivery channel may comprise a channel along which both: (i) light from the first light source, and (ii) light from the second light source, may travel as it is directed towards the subject's scalp, e.g. that channel is common to (e.g. used by) light from both light sources. The iNIRS system may comprise a plurality of interferometric optical detectors. Each detector may be coupled to the one or more reference channels for receiving reference light from the first and second light sources. Each optical detector may be arranged for receiving sample light from the subject's brain tissue.

The light emitting arrangement may comprise a light combiner arranged to combine light from each of the first and second light sources onto a common optical channel (e.g. so that light from both light sources will travel along that same common optical channel). The common optical channel may be coupled to the common sample delivery channel, or the common optical channel may be the common sample delivery channel. For example, light emitted from each light source may travel along the same stretch of optical channel as it travels towards the subject's scalp. The light combiner may comprise a wavelength division multiplexer. The light emitting arrangement may comprise a light splitter arranged to split light from the first and/or second light source between the common sample delivery channel and the one or more reference channels.

The light emitting arrangement may comprise: a first light splitter arranged to split light from the first light source onto a first sample delivery channel and a first reference delivery channel; a second light splitter arranged to split light from the second light source onto a second sample delivery channel and a second reference delivery channel; and a sample common light combiner arranged to combine light from the first sample delivery channel and light from the second sample delivery channel onto the common sample delivery channel. The light emitting arrangement may further comprise a reference common light combiner arranged to combine light from the first reference delivery channel and light from the second reference delivery channel onto a common reference delivery channel. The optical detector may be coupled to the common reference channel for receiving reference light therefrom. The first reference delivery channel may be of a different length to the second reference delivery channel. The light emitting arrangement may comprise: a common light combiner arranged to combine light from the first and second light sources, optionally wherein the common light combiner is arranged to couple the first and second light sources to a common optical channel; and a common light splitter coupled to the common light combiner and arranged to split light received from the common light combiner onto the common sample delivery channel and a common reference channel, optionally wherein the common light splitter is arranged to couple the common optical channel to the common sample delivery channel and the common reference channel.

The optical detector may be configured to: (i) combine sample light from the first light source with reference light from the first light source, and (ii) combine sample light from the second light source with reference light from the second light source. The iNIRS system may comprise a controller configured to determine an indication of scattering and/or absorption coefficients for sample light from the first light source and sample light from the second light source. For example, the controller may be configured to determine an indication of neural activity based on processed combined light signals. The controller may be configured to determine an indication of blood oxygen concentration based on the absorption coefficients for sample light from the first and second light sources. The controller may be configured to determine the indication of blood oxygen concentration based on blood absorption data indicating relative absorption coefficients for oxygenated and de-oxygenated haemoglobin at wavelengths of light in the first and second wavelength ranges. The controller may be configured to determine an indication of optical path lengths for sample light from the first and second light sources based on beat frequencies present in their associated combined light signals.

The controller may be configured to determine an indication of depth-resolved scattering and/or absorption coefficients for the subject's brain tissue based on determined scattering and absorption coefficients associated with different optical path lengths. The controller may be configured to determine an indication of depth-resolved blood oxygen concentration for the subject's brain tissue based on the determined depth-resolved absorption coefficients. The controller may be configured to determine an indication of a blood flow index for the subject for each of the first and second wavelength ranges. The controller may be configured to determine an indication of a total tissue oxygenation metabolism for the subject based on the determined blood flow index and the blood oxygenation concentration for the subject.

The light emitting arrangement may be configured to provide time division multiplexing for the emission of light from the first and second light source. The system may be configured to provide time multiplexing for operation of the first and second light sources so that operation of the first light source is temporally interleaved with operation of the second light source. The light emitting arrangement may be configured to provide wavelength division multiplexing for the emission of light from the first and second light source. An optical path length for reference light travelling to the optical detector from the first light source may be different to an optical path length for reference light travelling to the optical detector from the second light source such that beat frequencies associated with the first light source are separable from beat frequencies associated with the second light source. The difference in optical path length for reference light from the first and second light sources may be selected so that there is no, or minimal, spectral overlap between: (i) beat frequencies for sample and reference light from the first light source, and (ii) beat frequencies for sample and reference light from the second light source. A difference between: (i) an expected optical path length for sample light travelling to the optical detector from the first light source, and (ii) an optical path length for reference light travelling to the optical detector from the first light source, may be different to a difference between: (iii) an expected optical path length for sample light travelling to the optical detector from the second light source, and (iv) an optical path length for reference light travelling to the optical detector from the second light source, to inhibit spectral overlap between beat frequencies associated with the first and second light sources. The system may be arranged to provide a wavelength dependent delay in one of a common sample channel and a common reference channel.

The light emitting arrangement may comprise a wavelength division multiplexer and the light detecting arrangement comprises a wavelength division demultiplexer. The light emitting arrangement may be configured to provide emission of both light from the first light source and light from the second light source, wherein said emission is wavelength multiplexed. The light detecting arrangement may be configured to wavelength demultiplex the combined light signals so that combined light signals associated with the first light source are processed separately to combined light signals associated with the second light source. The optical detector may comprise a detection light splitter arranged to split: (i) light from the first light source onto a first detection channel, and (ii) light from the second light source onto a second detection channel. The detection light splitter may be arranged to provide: (i) a first combined light signal to the first detection channel, the first combined light signal comprising one or more components at a beat frequency between the sample and reference light from the first light source, and (ii) a second combined light signal to the second detection channel, the second combined light signal comprising one or more components at a beat frequency between the sample and reference light from the second light source. The light detecting arrangement may comprise signal processing circuitry configured to process first and second combined light signals separately.

Each light source may have an associated optical amplifier to increase the output power for the light emitted from that light source. The iNIRS system may include a plurality of light delivery channels and an optical switch configured to selectively direct light from the first and second light sources to each of the plurality of light delivery channels. The light emitting arrangement may include at least one common light channel comprising a single channel coupled to (e.g. directly or indirectly) each of the first and second light source to receive light therefrom. Light delivered to the subject's scalp from both light sources may travel along this same single channel. Similarly, reference light from both light sources may travel to the one or more detectors along at least one common light channel.

In an aspect, there is provided an interferometric near infrared spectroscopy, iNIRS, method (e.g. for neuroimaging and analysis) comprising: operating a first light source to provide wavelength-swept emission of light through a plurality of different wavelengths in a first wavelength range (e.g. above an oximetry isosbestic wavelength); operating a second light source to provide wavelength-swept emission of light through a plurality of different wavelengths in a second wavelength range different to the first wavelength range (e.g. below an oximetry isosbestic wavelength); directing light from each of the first and second light sources on to a common sample delivery channel and towards a subject (e.g. towards their scalp); directing reference light from the first and/or second light source onto one or more reference channels; combining, at an interferometric optical detector, reference light received from the one or more reference channels and sample light received from the subject (e.g. from their brain tissue), the sample light comprising light delivered towards the subject (e.g. towards their brain tissue) through the common sample delivery channel, to provide combined light signals comprising one or more components at a beat frequency between sample and reference light; and processing the combined light signals for providing imaging and analysis (e.g. of their brain).

Aspects of the present disclosure include one or more computer program products comprising computer program instructions to program a processor to control operation of an interferometric near infrared spectroscopy system to perform any methods disclosed herein.

Embodiments may provide iNIRS systems and methods for neuroimaging and analysis of a subject's brain tissue. The iNIRS systems and methods of the present disclosure are directed to a fundamentally different approach for performing neuroimaging and analysis, as compared to the fNIRS technologies described above. Embodiments may provide an improved approach for performing iNIRS neuroimaging and analysis. As disclosed herein, iNIRS systems of the present disclosure include two light sources and one or more light detectors. The iNIRS systems of the present disclosure may also include a controller arranged to receive output signals from the one or more light detectors.

Each light source may comprise a light generating element arranged to generate light (e.g. near infrared light). For example, each light generating element may comprise a laser. Each light source may comprise an optical arrangement coupled to the light generating element. The optical arrangement of each light source may be configured to deliver the generated light from the light generating element to each of one or more different locations. The optical arrangement of each light source may be arranged to direct some of the light from the light generating element towards a region to be sampled. The optical arrangement of each light source may be arranged to direct some of the light to each light detector. The optical arrangement of each light source may comprise a plurality of light delivery channels. The plurality of light delivery channels may include one or more sample delivery channels, and/or one or more reference delivery channels. Each light delivery channel may comprise an optical channel, such as an optical fibre. Each light delivery channel may be configured for transmitting light along its length (e.g. from the light generating element towards the subject's scalp or the light detector). The optical arrangement of each light source may comprise a light splitter for splitting light into each of the different delivery light channels.

The iNIRS system may be arranged so that, when installed on a subject's head (e.g. for providing neuroimaging and analysis of that subject's brain tissue), the optical arrangement of each light source is configured to direct some of the light towards the subject's scalp. For example, the optical arrangement of each light source may comprise a sample delivery channel (e.g. which is operable for directing sample light towards the subject's scalp). The iNIRS system may be arranged so that, in use, the optical arrangement of each light source may direct some of the light directly to the light detectors (e.g. for combining with sample light from the subject's brain tissue). For example, the optical arrangement of each light source may comprise a reference delivery channel (e.g. which is operable for directing reference light to one or more of the light detectors). The optical arrangement of each light source may be configured to deliver light from the light generating element to each light channel. The optical arrangement of each light source may be configured to deliver both: (i) light to the sample light delivery channel (‘sample light’), and (ii) light to the reference delivery channel (‘reference light’). For example, the optical arrangement of each light source may comprise a light splitter configured to split light received from the light generating element into each of the different channels.

The iNIRS system may be arranged so that, in use when installed on a subject's brain tissue, the sample light may be directed towards the subject's scalp and brain tissue (e.g. through the sample delivery channel), and the reference light may be directed towards each light detector (e.g. through the reference delivery channel). Each light source may be arranged to provide wavelength swept emission of light (e.g. each light source may be arranged to output light at each of a plurality of different wavelengths in a selected time period). For example, each light source may comprise a modifying element for controlling operation of the light generating element to output light at each of a plurality of different wavelengths. Each light source may be arranged to sweep the wavelength of the light it outputs (e.g. increasing or decreasing in wavelength). Each light source may be arranged to provide chirped emission of light in which, each chirp (or ‘pulse’) comprises one wavelength sweep. Each light source may be arranged to output sequential chirps with the same wavelength sweep, e.g. such that the wavelength of the light output from the light source changes according to a repeating pattern.

Each light detector may provide an interferometric optical detector. Each light detector may comprise an optical arrangement. The optical arrangement of the light detector is configured to direct light to be detected into the light detector (e.g. from the subject's scalp). The optical arrangement of the light detector may comprise a plurality of light receiving channels. The plurality of light receiving channels may include one or more sample light receiving channels, and/or one or more reference light receiving channels. Each light receiving channel may comprise an optical channel, such as an optical fibre.

The iNIRS system may be arranged so that, when installed on a subject's head (e.g. for providing neuroimaging and analysis of that subject's brain tissue), the optical arrangement of the light detector is configured to receive light emitted from the light source (e.g. which has travelled through the subject's brain tissue from the light source). For example, the optical arrangement of the light detector may comprise a sample receiving channel (e.g. which is operable for receiving sample light from each light source which has passed through the subject's brain tissue). The iNIRS system may be arranged so that, in use, the optical arrangement of the light detector may receive some of the light from each light source which has travelled directly from the light source (e.g. which has travelled along an optical channel). For example, the optical arrangement of the light detector may comprise a reference receiving channel (e.g. which is operable for receiving reference light from one or more light sources). Each light detector may be coupled to each light source so that the reference delivery channel of the light source is coupled to the reference receiving channel of the light detector (e.g. so that reference light may travel from the light generating element to the light detector via the reference delivery and receiving channels).

The optical arrangement of the light detector may be configured to deliver both to the light detector: (i) light from the sample light receiving channel (‘sample light’), and (ii) light from the reference receiving channel (‘reference light’). For example, in use, the detector is arranged to receive both: (i) sample light from each light source which has passed through the subject's brain tissue, and (ii) reference light from each light source which has travelled to the light detector along one or more reference channels.

The light detector may be arranged to combine reference light with sample light to provide a combined light signal. For example, the light detector may comprise a light combiner (e.g. for combining light on the reference receiving channel with light on the sample receiving channel). The combined light signal may include a plurality of components at beat frequencies, e.g. at frequencies corresponding to the differences in wavelength between the sample light and the reference light. Each light detector is configured to convert received combined light signals into one or more electrical signals indicative of that combined light signal. For example, the detector may comprise one or more photodiodes. Each photodiode may output an electrical signal (e.g. a current) indicative of the combined light signal. The detector may comprise a balanced photodetector (e.g. which includes two photodiodes, which may be 180° out of phase with each other, and its output may be a combination of the two photodiode current outputs). The detector may optionally include current to voltage conversion circuitry and/or one or more amplifiers for amplifying the electrical signal.

The iNIRS system may include at least one analogue to digital converter arranged to convert electrical signals representing the sample light (e.g. the combined light signals) into one or more digital signals. The controller is arranged to process the digital signals to determine one or more properties of the subject's brain tissue. The controller may be configured to determine optical properties of the subject's brain tissue (e.g. for absorption and/or scattering). The controller may be configured to determine one or more dynamic properties of the subject's brain tissue (e.g. properties of the subject's brain tissue which are varying over time). For example, the controller may be configured to detect the presence of movement within the subject's brain tissue (e.g. due to movement, such as flow, of blood within the brain tissue).

The controller may be configured to process the digital signals to obtain time of flight information for photons of sample light travelling from each light source through the subject's brain tissue to the light detector. The controller may be configured to identify penetration depths (and optionally expected trajectories for photons through the brain tissue) associated with the different times of flight for sample light photons. The controller may be configured to obtain a time-ordered series of time of flight distributions for sample light photons reaching each light detector. The controller may be configured to process the time-ordered series to identify changes in the time of flight distribution over time, such as identifying decay and/or decay rates between success time of flight distributions. The controller may be configured to provide depth-resolved processing, e.g. by filtering the time of flight data to focus on only photons within a selected time of flight range (e.g. to identify changes in optical properties of the brain tissue for penetration depth(s) associated with that time of flight range). The controller may be configured to process data received from the light detector(s) to provide time of flight information with depth-resolved autocorrelations for the subject's brain tissue.

The controller may be configured to process the received data indicative of sample light received at a light detector and to output a control signal based on that received data. The control signal may provide an indication of the time of flight distribution (e.g. the controller may be configured to output the time of flight distribution). The control signal may provide an indication of one or more properties determined based on the time of flight distribution, such as optical properties for the brain tissue (e.g. scattering and/or absorption coefficients, and/or how these have changed/are changing). The control signal may provide an indication of blood flow within the subject's brain tissue. The control signal may provide a depth-resolved indication of one or more properties of the subject's brain tissue (e.g. linked to a specific region within their brain tissue, such as at a selected penetration depth range). The control signal may comprise an indication of one or more properties of the subject's brain tissue, such as intracranial pressure, blood flow index, artery elasticity, cerebral metabolic rate of oxygen consumption. The medical properties may be associated with specific regions/depths within the subject's brain tissue. The control signal may comprise an actuation command for a brain-computer interface, e.g. to control operation of a device based on the actuation command. The control signal may comprise an image for display, where that image represents a portion of the subject's brain tissue (as determined based on the received sample light).

### FIGURES

Some examples of the present disclosure will now be described, by way of example only, with reference to the figures, in which:

FIG. 1 shows a schematic diagram of an iNIRS system.

FIGS. 2a and 2b show schematic diagrams of arrangements for delivering light to sample and reference delivery channels.

FIGS. 3a, 3b, 3c and 3d show schematic diagrams of arrangements for receiving light at a detector from sample and reference receiving channels.

FIG. 4 shows an example delay arrangement for a common optical channel.

In the drawings like reference numerals are used to indicate like elements.

### SPECIFIC DESCRIPTION

Embodiments of the present disclosure are directed to systems and methods for non-invasively determining an indication of blood oxygenation within a subject's brain tissue. An interferometric near infrared spectroscopy (‘iNIRS’) system is used which includes a first light source and a second light source. One of the light sources emits light at wavelengths above an oximetry isosbestic wavelength, and the other of the light sources emits light at wavelengths below the oximetry isosbestic wavelength. Each light source will provide wavelength swept emission. The iNIRS system also includes one or more light detectors, where each light detector is arranged to receive sample and reference light from both of the two light sources. Each light detector interferometrically combines sample and reference light from each of the two light sources to provide combined light signals comprising components at one or more beat frequencies between sample and reference light. Scattering and/or absorption coefficients may be determined by based on the combined light signals. A blood oxygenation level may be determined for the subject's brain tissue based on differences in absorption between light from the two different light sources. Depth-resolved blood oxygenation levels may be determined by monitoring coefficients for photons associated with different beat frequencies (and thus different photon times of flight/penetration depth).

### Interferometric Near Infrared Spectroscopy (‘iNIRS’)

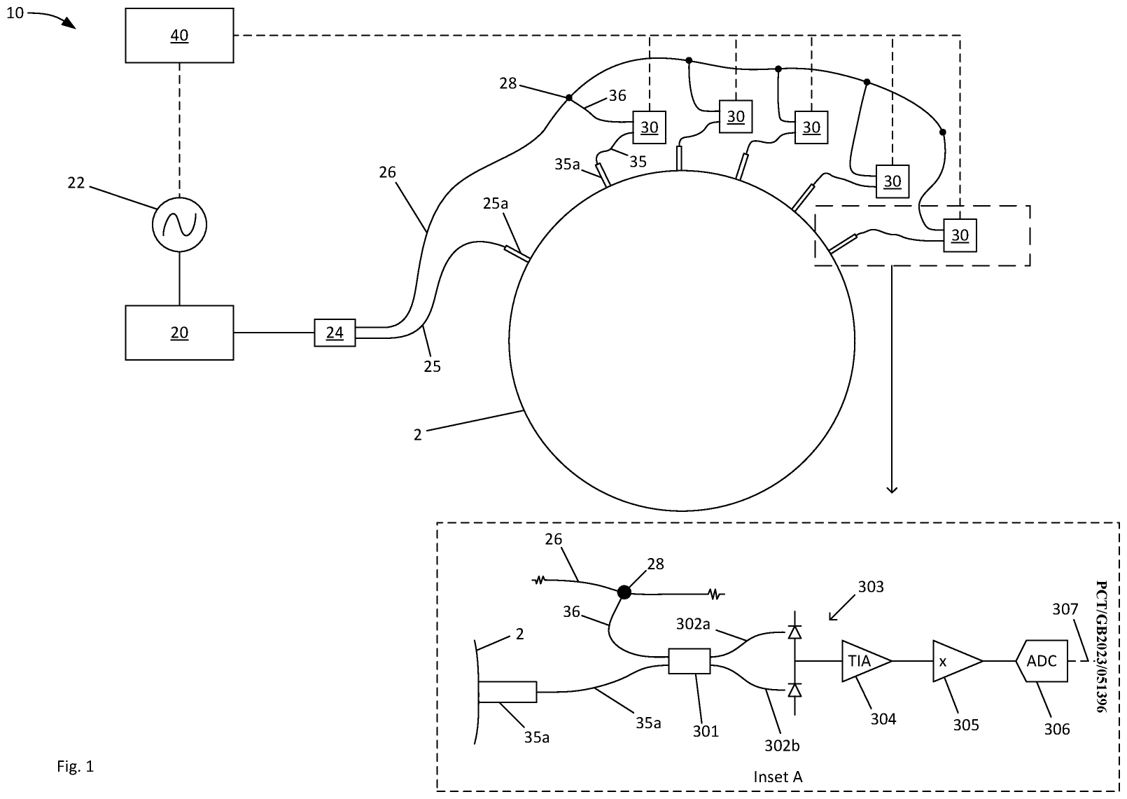

FIG. 1 shows a schematic diagram of an interferometric Near Infrared Spectroscopy (‘iNIRS’) system 10. The iNIRS system 10 includes a light source 20, a plurality of light detectors 30, and a controller 40. Inset A of FIG. 1 shows a more detailed view of one of the light detectors 30.

The iNIRS system 10 includes a light source modifier 22, and a light splitter 24. The iNIRS system 10 includes a sample delivery channel 25 and a reference delivery channel 26. The iNIRS system 10 is shown coupled to a subject's head 2. The iNIRS system 10 includes a sample delivery probe 25a and a plurality of sample receiving probes 35a. For each light detector 30, there is an associated sample receiving probe 35a, a sample receiving channel 35, a reference delivery channel connection 28, and a reference receiving channel 36.

The light source modifier 22 may comprise a source for providing a variable electrical control signal (e.g. a variable current or voltage provider). The light source modifier 22 is coupled to the light source 20. The light source modifier 22 may be electrically connected to the light source 20 to provide a variable current/voltage thereto.

The light source 20 may comprise a laser. For example, the laser may be a Distributed Feedback laser (‘DFB’) or a MEMS-Vertical Cavity Surface Emitting laser (‘MEMS-VCSEL’). The light source 20 is coupled to the light splitter 24. The light splitter 24 has an input for receiving light from the light source 20. The light splitter 24 has two outputs for transmitting light from the light source 20 to two separate channels. The sample delivery channel 25 is coupled to the light splitter 24 (to receive light therefrom), as is the reference delivery channel 26. The sample delivery channel 25 couples the light splitter 24 to the sample delivery probe 25a. The sample delivery probe 25a will be placed at a location on the subject's scalp.

Other types of suitable laser include a Distributed Bragg Reflector laser (‘DBR’), a Fourier Domain Mode Locking laser (‘FDML’), a Vertical Cavity Surface-Emitting laser (‘VCSEL’). Additionally, or alternatively, a pulsed supercontinuum laser may be used in combination with a pulse stretching mechanism, such as a grating or GRISM pulse stretcher or length of dispersive optical fibre. For example, such an arrangement may be configured to temporally separate the wavelengths in the pulse such that a frequency chirped pulse is created (e.g. for ultimately providing an interferogram when sample and reference pulses are compared).

The reference delivery channel 26 couples the light splitter 24 to each of the light detectors 30. For each detector, the reference delivery connection 28 couples the reference delivery channel 26 to the reference receiving channel 36 for that detector. Each reference receiving channel 36 is coupled to its light detector 30. Each light detector 30 is connected to the light source 20 to directly receive reference light therefrom (via one or more reference channels). Each sample receiving probe 35a is placed on the subject's scalp. Each sample receiving probe 35a is coupled to the sample receiving channel 35. Each sample receiving channel 35 is coupled to its light detector 30. Each light detector 30 is connected for indirectly receiving sample light from the light source 20 (via the sample delivery and receiving channels, and via the subject's brain tissue therebetween).

There are a plurality of different light detectors 30. Each detector may comprise an interferometer, such as a Mach-Zehnder interferometer. Each of the different light detectors 30 is coupled to the same light source 20 (each via one or more reference channels). The light detectors 30 are spatially separated from the light source 20. The light detectors 30 may also be spatially separated from one another or they may be co-located on a sufficiently similar region of tissue that the received signals can be averaged together. For reference light to reach the light detector(s) 30 from the light source 20, the reference light will travel directly along one or more reference channels. For sample light to reach the light detector 30 from the light source 20, the sample light will travel indirectly via the subject's brain tissue. The sample light is delivered to the subject's scalp via one or more delivery channels. The sample light may then pass through the subject's brain tissue where it will be received and transmitted to a light detector 30 via one or the sample receiving channels 35. The illumination of the subject's brain tissue may thus occur using a different light channel to the detection of light from the subject's brain tissue.

The controller 40 may comprise any suitable component with data receiving and processing functionality. For example, the controller 40 may include at least one Application Specific Integrated Circuit (‘ASIC’). Other examples for the controller 40 may include a Field Programmable Gate Array (‘FPGA’) and/or a Data Acquisition module (‘DAQ’). The controller 40 is coupled to each of the detectors. The controller 40 may be connected to each detector via a wired connection (for receiving electrical signals indicative of detection therefrom), and/or the connection may be wireless (for receiving transmitted data indicative of detection therefrom). The controller 40 is coupled to the light source modifier 22. This connection may be wired or wireless.

The iNIRS system 10 may be at least partially housed within a garment for the subject's head 2. For example, the iNIRS system 10 may be provided in a hat/cap which is to be worn by the subject on their head 2. The head garment may be arranged to hold the light source 20 and detectors in a fixed arrangement relative to the subject's scalp. Some or all of the components may be provided with the head garment. For example, the head garment may include a plurality of receiving portions for receiving light source(s) 20 and light detectors 30. Channels connecting the light sources and light detectors 30 may be provided as part of the head garment (e.g. they may be routed through corresponding channel receiving portions of the head garment). The controller 40 may be separate to the head garment (e.g. and connected wirelessly) or it may also be provided as part of the head garment (e.g. by an ASIC within the head garment which may be wire coupled to the detectors and/or light source modifier 22). For example, the garment may be configured to receive the source and detection channels and the probes, with the other components of the system located elsewhere.

Some or all of the channels of the iNIRS system 10 may be provided by optical fibres. Light splitters of the present disclosure may comprise fibre-optic splitters. The iNIRS system 10 may include lenses, reflection and/or refraction devices for beam steering, as relevant. For example, the sample delivery probe 25a may include one or more lenses for spatially distributing sample light from the sample delivery channel 25 towards the subject's brain tissue. As another example, one or more of the sample receiving probes 35a may include a lens for focussing received light into the sample receiving channel 35 connected to that sample receiving probe 35a. As another example, the probes may be bare fibres which have been cleaved and/or polished.

The iNIRS system 10 is arranged to provide a plurality of source-detector pairs for each light source 20. In other words, the iNIRS system 10 is arranged so that each light detector 30 may receive two forms of light: (i) reference light, and (ii) sample light. Each detector is arranged to receive reference light directly from the light source 20 (the reference light will travel from the light source 20 along one or more channels to the light detector 30, e.g. without passing through the subject's brain tissue). Each detector is also arranged to receive sample indirectly from the light source 20 (the sample light will have been directed towards the subject's scalp tissue and a portion may have travelled through their brain tissue en route to the detector, e.g. the sample light will not have travelled exclusively through optical channels between the light source 20 and light detector 30).

The detectors 30 are arranged to be positioned on the subject's scalp to provide imaging of a selected region of their brain. At least some of the detectors 30 are arranged to be spatially separated from the light source 20. One or more (e.g. each) of the light detectors 30 may be arranged to be sufficiently spaced apart from the light source 20 so that at least some of the photons of sample light from the light source 20 which is received at the light detector 30 will have penetrated into the subject's brain tissue. For example, the source-detector spacing may be selected so that the light detector 30 is arranged to receive sample light photons which have undergone multiple scattering events (e.g. which have scattered multiple times between source and detector as they travel through the subject's head 2). In other words, the source-detector spacings may be selected so that light detectors 30 are receiving deeply penetrating photons from the subject's brain tissue. Such photons may have longer time of flights from source to detector, as compared to photons which penetrate more shallowly and undergo fewer scattering events.

The detectors 30 may be arranged to be arranged to be spatially proximal to each other on the subject's scalp. The arrangement of the detectors 30 may be selected so that the detectors are imaging a similar region of the subject's brain. For example, the detectors 30 may be located within a threshold distance of each other on the subject's scalp so that they data they obtain may be averaged (e.g. to provide average values for the same volume of the subject's brain). That is, the detectors 30 may be arranged to spatially probe the same volume of tissue within the subject's brain. For example, the detectors may be arranged to be within one attenuation length for the tissue of each other (e.g. within the sum of the absorption and scattering coefficients).

The light source 20 is arranged to generate light and to direct this light towards the subject's scalp and the light detectors 30 (via the reference channel(s)). The light splitter 24 is arranged to receive light generated by the light source 20 and to split this light into two channels: (i) towards the subject's scalp using the sample delivery channel 25 and sample delivery probe 25a, and (ii) to the light detectors 30 using the reference delivery channel 26 and reference receiving channels 36. The splitter is configured so that the majority of the light is directed towards the subject's scalp. For example, the splitter may be a 90:10 splitter, or a 99:1 splitter. The sample delivery channel 25 is arranged to receive sample light from the splitter, and to deliver this sample light towards the subject's scalp (via the sample delivery probe 25a). The reference delivery channel 26 is arranged to receive reference light from the splitter, and to deliver this sample light to the detectors (via the reference receiving channels 36).

Each of the reference delivery connections 28 is arranged to deliver some of the reference light travelling along the reference delivery channel 26 to one of the reference receiving channels 36. Each of the reference receiving channels 36 is arranged to deliver the reference light to its light detector 30. The sample receiving probe 35a is arranged to receive sample light from the subject's brain tissue. The sample receiving probe 35a may focus the received sample light onto the sample receiving channel 35. The sample receiving channel 35 is arranged to deliver received sample light to its light detector 30. The sample receiving probes 35a may be arranged in close proximity to each other on the subject's scalp.

Each detector is arranged to receive two inputs: (i) reference light directly from the light source 20, and (ii) sample light indirectly from the light source 20 (e.g. which has travelled via the subject's brain tissue, as well as through their scalp skin and skull). For example, each detector may comprise two or more input ports. A first input port of the detector may be coupled to the reference delivery channel 26 for that detector. A second input port of the detector may be coupled to the sample delivery channel 25 for that detector. The detector is arranged to combine reference light with sample light (as an interferometer). The detector and controller 40 are arranged to determine one or more properties of the subject's brain tissue based on this combination of reference light and sample light (as will be described in more detail below).

The light source 20 is configured to provide wavelength swept emission of light. For this, the light source 20 may be configured to produce a series of emissions of pulses of light. During each pulse, the wavelength of light may be “swept” through a range of wavelengths. For example, the sweeping may be in the form of a chirped pulse. Light will be emitted at a plurality of different wavelengths during one pulse. For example, the wavelength may continually increase or decrease during one pulse (the rate of change of wavelength may be constant, or it may be variable). The series of chirped pulses may be contiguous (e.g. with a zero inter-pulse time interval). The light source 20 may be configured to successively emit a series of pulses, with each pulse having a wavelength sweep. However, it will be appreciated that the light source 20 need not provide continuous sweeping. For example, the light source could be tuned in steps rather than continuously, such that the light source 20 emits light at different wavelengths in different time intervals (e.g. discrete time intervals for emission at each of a plurality of wavelengths). The light source 20 may sweep unidirectionally (e.g. only increasing or decreasing in wavelength during one wavelength sweep), or it may sweep bidirectionally (e.g. both increasing and decreasing in wavelength during one wavelength sweep).

The controller 40 may be configured to selectively control the wavelength sweeping of the light source 20. The light source modifier 22 is arranged to control the wavelength emission of light from the light source 20. For instance, the light source modifier 22 may be arranged to apply a selected current (or voltage) to the light source 20 to select a wavelength emission from the light source 20. The wavelength sweeping of the light source 20 may be controlled by using the light source modifier 22 to apply a corresponding electrical signal to the light source 20. The controller 40 may be arranged to control application of a current/voltage to the light source 20 using the light source modifier 22 to provide a selected pattern for the wavelengths of light emitted by the light source 20.

The light source 20 may be controlled to wavelength sweep according to a selected pattern for the sweeping. For example, the light source 20 may sweep through a selected range of wavelengths of light and/or the light source 20 may sweep through wavelengths of light according to a selected sweep profile (e.g. linear increasing, sinusoid, triangular etc.). For example, the light source 20 may sweep according to a selected sweeping rate, or a selected total sweeping time. The light source 20 is configured to wavelength sweep light so that during one wavelength sweep, light will be directed towards the subject's brain tissue through the sample delivery channel (and to the detectors via the reference channels) at each of a plurality of different wavelengths. The wavelength of light emitted by the light source 20 will vary over time. As such, an indication of the time at which light was emitted from the light source 20 may be determined based on a wavelength of that light.

The light source 20 may be configured to sweep through a selected wavelength range. For example, the light source 20 may be configured to sweep in optical frequency over a range of 50 GHz. For example, this may enable the light source 20 to emit modulated light at a plurality of different wavelengths between e.g. 829.94 nm and 830.06 nm when centred on 830 nm for example or between 1309.857 nm and 1310.143 nm when centred on 1310 nm for example. The light source 20 may be configured to sweep through a wavelength range of at least 0.025 nm, such as at least 0.05 nm, such as at least 0.075 nm, such as at least 0.1 nm, such as at least 0.11 nm (e.g. about a wavelength on which it is centred). The light source 20 may have a high output power, a long coherence time, and broad mode-hop free wavelength tuning. The light source 20 may have a relatively narrow linewidth and a longer coherence length, e.g. because the light source 20 will not sweep over particularly large bandwidths.

Light sources of the present disclosure may be configured to provide emission of high coherence light, e.g. substantially coherent light. It will be appreciated that the light source may not both emit perfectly coherent light and also provide wavelength swept emission of light, e.g. because light at different wavelengths will change phase at different rates. Light sources of the present disclosure may be controlled to sweep through a wavelength range which is relatively narrow compared to their absolute wavelength. In other words, the difference between the maximum and minimum wavelengths for one wavelength sweep will be relatively small compared to those wavelengths. Each light source may be configured to emit light (i.e. an electric field) which does not have much change in its phase over time.

The iNIRS system of the present disclosure will receive sample light and reference light, both of which originated from the same light source. The light sources of the present disclosure are configured to provide wavelength swept emission of sufficiently coherent light, such that the sample light and reference light, as received at the optical detector, will be in relatively similar phase to each other. As such, the combination of sample and reference light will give rise to substantially constructive interference between the two waves (e.g. the two streams of light wave will have sufficiently similar phases so that the resulting combined light signal will contain a constructive combination of the two light waves). In other words, the coherence length of the light source may be such that the multiple scattering in the tissue will not reduce the coherence or fringe contrast below a noise floor for the measurement.

For example, each light source of the present disclosure may comprise a laser. The laser may be selected based on its coherence length, e.g. to enable the constructive interference described above between sample light and reference light to occur. In other words, the iNIRS system may be arranged so that a maximum expected time of flight delay for sample light photons (received at the optical detector which have travelled through the subject's brain tissue) relative to reference light photons (received at the optical detector which have travelled along the one or more reference channels) is within a coherence time period for the laser (e.g. the difference in optical path length between the sample and reference light is within the coherence length of the laser). Within this coherence time period, the phase of light emitted by the laser is approximately stable (despite changes in the wavelength of light being emitted). As such, there may be no loss in amplitude for combined light signals at the optical detector (e.g. the interference occurring at the detector may be substantially completely constructive).

For example, the iNIRS system may be configured to have a coherence length or range of approximately 50 m in air—e.g. the light sources may be selected which have a coherence length of between 50 and 100 m (a coherence time period of between 166 ns and 333 ns). It will be appreciated that this particular range is not intended to be limiting, rather it is illustrative of the approximate range for the light source. The light source may be selected so that it has a coherence length which is two or more times greater than the maximum expected optical path length difference, e.g. the coherence length may be three or four or more times greater. Having a light source with a coherence length which is much greater than the optical path length may increase accuracy for measuring sample light photons which have undergone a large number of scattering interactions within the subject's brain tissue.

The iNIRS system 10 is arranged so that the source-detector path lengths for reference and sample light are different. In other words, the iNIRS system 10 is arranged so that an average, or expected, optical path length for light travelling from the light source 20 to each detector via the subject's brain tissue will be different to the optical path length for light travelling from the light source 20 to said detector via reference channel(s).

As will be appreciated in the context of the present disclosure, photons of sample light which are directed towards the subject's brain tissue may travel from the light source 20 to a light detector 30 via a practically infinite number of different paths. A photon of sample light may undergo a large number of scattering events, and so follow a very tortuous path, between the sample delivery probe 25a and the sample receiving probe 35a. The iNIRS system 10 is arranged to provide neuroimaging and analysis based at least in part on activity in the subject's brain tissue. As such, photons which do not penetrate into the brain tissue (e.g. which are scattered at shallow depths, such as skin, skull, membrane) may carry little to no information as to blood oxygenation levels in the subject's brain. The reference paths may be selected so that they are of a shorter source-detector path length than such a null information sample light photon path length. As compared to this null information sample light photon path length, photons which penetrate deeper into the subject's brain tissue (and which may thus carry potential useful information) will typically traverse a longer pathlength from light source 20 to light detector 30. The time of flight for a sample light photon from light source 20 to light detector 30 will of course increase as the path length it takes increases. As such, a photon which travels a longer path, and penetrates deeper into the subject's brain tissue, will take even longer to arrive at the light detector 30. The longer the time of flight for a sample light photon, the deeper that photon is likely to have penetrated into the subject's brain tissue.

The iNIRS system 10 is arranged so that the shortest time of flight for photons of light to travel from light source 20 to light detector 30 will be for photons of reference light travelling along the reference channel(s). The sample light photons will have longer times of flight than this reference light. The sample light photons which penetrate the deepest into the subject's brain tissue are likely to be those which have the longest time of flight to the light detector 30.

The iNIRS system 10 is arranged to determine a distribution for time of flight (‘DTOF’) for sample light photons. For this, a temporal point spread function (‘TPSF’) may be determined, which is similar to the DTOF, but which includes an Instrument Response Function (‘IRF’) which can subsequently be filtered out (e.g. deconvolved and/or subtracted) using post-processing. Each determined DTOF may provide a distribution showing the time of flight for all sample light photons which were incident on the light detector 30 at a given moment in time. The DTOF may contain an ensemble average representing a large number of incident photons (in each of a plurality of different TOF bins). The intensity for each TOF bin will provide an indication of the number of incident photons at that TOF. A phase of a TOF bin (e.g. obtained using a Fourier analysis) may represent an average phase for all of the photons arriving in that TOF bin. As described in more detail below, numerous properties of the subject's brain tissue may be determined based on DTOF data obtained for the subject's brain tissue. To obtain such data, the iNIRS system 10 is configured to obtain interferograms for the combination of reference light and sample light, as received at a given light source 20.

The iNIRS system 10 is arranged so that each of the light detectors 30 receives both sample light and reference light, and combines the two using a light combiner. For example, each of the detectors may provide an interferometer assembly (in combination with the sample and reference light channels) configured to combine the reference light and the sample light to obtain an interference pattern (an interferogram).

The light source 20 is configured to emit substantially coherent light. The resulting interference pattern for light from the light source 20 (as obtained at each detector) may comprise a combined signal having components at beat (or intermediate/difference) frequencies corresponding to the difference in wavelength between: (i) wavelengths of the photons of sample light received at the light detector 30 at a given instance in time, and (ii) the wavelength of photons of reference light received at the light detector 30 at that instance in time. The reference light at a single sampling interval should be substantially narrow and uniform wavelength, as limited by either the intrinsic lineqidth of the laser and the optical frequency sweeping rate, as the received photons of reference light will have travelled the same distance (through reference channels) to the light detector 30 from the light source 20. The sample light will include photons at different wavelengths, where each wavelength of sample light will correspond to the time of flight for that photon and its unique path through the tissue (due to the wavelength sweeping of the light source 20). The resulting interferogram will therefore contain a plurality of different beat frequencies (due to the different differences in wavelength). The higher beat frequencies may correspond to photons with higher times of flight (deeper penetrating photons) in the event that the sample path is longer than the reference path.

Each light detector 30 comprises a light combiner arranged to combine the light, and to provide said combined light (which may include one or more components corresponding to beat frequencies) to signal processing circuitry. Each of the detectors may comprise a square law detector. For example, each detector may be configured to create an interference pattern based on the difference in optical frequencies of the incident sample and reference electric fields, e.g. wherein the intensity or power detected is proportional to the square of the incident electric field, and the incident electric field is the sum of the sample and reference electric fields. Such that the intensity or power detected (in the form of a photocurrent) is equal to the square of the sum of the incident sample and reference electric fields. Such detectors may comprise photodiodes, avalanche photodiodes and/or fast linescan cameras, streak cameras and fast CCD or CMOS sensors. The detector may be a high bandwidth detector. For example, the detector may be configured to resolve interference fringes at 100 Mhz or more, such as up to 1 GHz. The detector may comprise a single speckle detector. For example, optical fibres used in the detector may be single mode. If a multi-mode detector is used, then the detector may comprise an array of square-law detectors, such as a photodiode array, a focal plane array, fast linescan camera or fast CCD array. The detector may also comprise a balanced detector array. For example, the balanced detector array may be configured so that the reference light and the scattered light are combined and split (e.g. evenly) onto a pair of out-of-phase detectors such as with a 4-port (2-in, 2-out 50:50 ratio) fibre coupler or a beamsplitter cube. A balanced detector may enhance signal to noise of the detected signal by rejecting incoherent portions of the signal. The balanced photodetector may also fully utilise all light transmitted through the interferometer and suppress common noise, such as laser intensity noise.

In other words, each light detector 30 may comprise a light combiner configured to combine the sample and reference light to provide a combined light signal. The iNIRS system 10 is arranged to process that combined light signal to obtain an indication of the intensity of light incident on the light detector 30 at a given moment in time (e.g. through use of optical heterodyning and/or balanced detection). The iNIRS system 10 is arranged to obtain a plurality of such indications, e.g. the light detector 30 may be arranged to repeatedly obtain indications of the intensity of light incident on the light detector 30. In other words, the iNIRS system 10 is arranged to measure a phase or frequency shift between photons of light in the two inputs to the detector (reference and sample), and to attribute such differences to properties of the intervening brain tissue for the sample light.

Each obtained interferogram may be Fourier analysed (e.g. using an FFT or IFT) to obtain an indication of a DTOF for the sample light photons incident on the light detector 30. When a square-law detector is used, the intensity at the detector may be proportional to the square of the summed electric field intensity. The rate of change of the optical frequency multiplied by time delay may give rise to a frequency of the interference fringes present in each interferogram. In other words, a Fourier Transformed interferogram containing a plurality of beat frequencies may be used to indicate the photon time of flights associated with those beat frequencies present in the interferogram.

To obtain this data of DTOF for sample light photons, the light detector 30 is arranged to combine the two light inputs (sample and reference) into a combined light beam. The light detector 30 is arranged to convert the combined light beam into an electrical signal representative of the combined light signal. The iNIRS system 10 includes at least one digitiser arranged to receive electrical signals indicative of combined light signals and to convert those electrical signals into digital data representing said combined light signals. This digital data may be processed to obtain one or more different properties of the subject's brain tissue for neuroimaging and analysis.

One example of an arrangement for converting received light signals into digital data is shown in Inset A of FIG. 1. Inset A shows an arrangement of components that may be used as a light detector 30 of the present disclosure. As also shown in the iNIRS system 10 of FIG. 1, the detector 30 receives two inputs: (i) reference light which has travelled along reference delivery channel 26 and reference receiving channel 36, and (ii) sample light which has been received through the sample receiving probe 35a and delivered to the detector via the sample receiving channel 35.

As shown, the detector may include a light combiner and splitter 301, a first light channel 302a and a second light channel 302b, a balanced photodetector 303, a transimpedance amplifier 304, an amplifier 305, an analogue to digital converter (‘ADC’) 306. The ADC 306 is arranged to provide a digital signal output 307.

The light combiner and splitter 301 is coupled to both the reference receiving channel 36 and the sample receiving channel 35. The light combiner and splitter 301 is arranged to receive both the sample and reference light, and to combine the two to provide a combined light signal. The light combiner and splitter 301 is arranged to split that combined light signal onto two separate channels: the first light channel 302a and the second light channel 302b. For example, this may be a 50:50 split (or there or thereabouts). The first light channel 302a and second light channel 302b are coupled to a balanced photodetector 303. Each light channel directs light towards an associated photodiode. The balanced detector is arranged to provide an output based on a difference between outputs from the two photodetectors. The two photodetectors will typically be provided so that the beat signals on each photodiode are 180° out of phase with each other, and so the coherent AC terms will combine positively with each other. The balanced photodetector 303 is arranged to output a current corresponding to the difference between the two photodetector output currents. The balanced photodetector 303 may remove any unwanted DC terms from this signal, such as slow fluctuations emanating from the light source 20 or other common-mode effects such as noise.

The light detector 30 is configured to use a current to voltage converter to convert the current output from the balanced photodetector 303 into a corresponding voltage. As shown in Inset A of FIG. 1, the converter may comprise a transimpedance amplifier 304. The voltage output from the transimpedance amplifier 304 is then amplified using the amplifier 305. The amplifier may be used to scale the output signal to the full range of the ADC and limit the electronic frequency of the circuit to further maximise the SNR. This amplified voltage is then provided to the ADC 306 to be digitised. The ADC 306 comprises a digitiser having sufficient bandwidth so that the full signal bandwidth containing time of flight information may be digitised without attenuation. For example, the digitiser bandwidth may be at least as large as the bandwidth for the combined light signal. For example, the digitiser may be selected to have a sampling rate high enough so that the Nyquist criterion is met for the bandwidth of the signal to be processed. The digitiser may be provided as part of each light detector 30, or the digitiser may be part of the controller 40, and the controller 40 may be coupled to each of the light detectors 30 to receive electrical signals therefrom which are to be digitised. For each combined light signal, a digital signal output 307 will be provided which gives a digital representation of that combined light signal (and thus of the sample light incident on the light detector 30 at the moment in time when that combined light signal was generated and measured).

The iNIRS system 10 is configured to obtain a plurality of digital signal outputs 307 indicative of sample light incident on light detectors 30. In particular, each light detector 30 is configured to repeatedly combine light signals (sample and reference) for providing digital signal outputs 307 representative of each combined light signal. For example, for each light detector 30, a time series of digital signal outputs 307 may be obtained, wherein each subsequent digital signal output 307 is for a subsequent point in time at which a combined light signal was obtained and measured (and the digital signal output 307 represents that combined light signal as obtained and measured). As described above, each of these signals may be indicative of a sample light DTOF for that point in time at that detector.

In other words, the iNIRS system 10 is configured to obtain a plurality of time-ordered DTOFs for each of a plurality of different light detectors 30. The digitiser may provide a digital output indicative of the different measurements, and this digital output may optionally be processed in a number of ways to provide the DTOF data. Examples of such steps will now be described.

The controller 40 may be configured to receive raw digital interferogram data (e.g. data representative of the interferogram obtained by converting the combined light signal into digital data). This raw interferogram may be divided into individual sweeps for the wavelength swept emission from the light source 20. For example, the sweep rate of the light source 20 and a time at which the first sweep commenced may be used to determine the sweep cycles. The data may then be divided into groups, where each group represents an individual sweep. An optional Hilbert transform may be performed on the data at this stage. Data windowing may be performed (e.g. with a Hann or Blackman-Harris window) to reduce sidelobes in the data. A Fourier analysis may be performed on the data, either inverse or normal. For instance, an inverse Fourier Transform may be performed. The Fourier analysis may be performed for each wavelength sweep of the light source 20. The resulting data may be in the form of a series of Temporal Point Spread Functions (‘TPSF’), with each TPSF corresponding to an associated wavelength sweep. The TPSF data may be processed to remove an Instrument Response Function (‘IRF’) therefrom to provide the DTOF data.

In other words, the iNIRS system 10 may be configured to determine a time-ordered series of time of flight distributions for sample light photons incident on each of a plurality of different detectors. This DTOF data may be processed to provide information relating to a number of different physical properties of the subject's brain tissue. The DTOF data may be used to determine optical properties of the medium through which the sample light has travelled. Each TOF bin in a DTOF may represent a selected volume within the subject's brain, and each DTOF represents a total volume of tissue probed by the photons (e.g. each DTOF may represent a weighted average of properties of the brain tissue, as well as other tissues through which the photons have travelled such as scalp, skull etc.). The optical properties include scattering and absorption properties for the subject's brain tissue. The DTOF data may be used to determine dynamic properties of the subject's brain tissue, such as how particular properties vary over time. This includes how the optical properties evolve over time, as well as properties indicative of movement within the subject's brain tissue (e.g. due to the flow of blood). The iNIRS system 10 may be configured to Fourier transform (e.g. FFT) the obtained TOF distributions to obtain gamma data, where the dynamical signals may be obtained from temporal changes in the gamma signals.

The time-ordered series of DTOFs may be considered to correspond to a surface of data in a 3D volume. That surface may represent the DTOF for each subsequent DTOF (ordered in time), and so the surface shows each individual DTOF, as well as the evolution of the DTOFs over time. This surface may provide a wealth of data from which properties of the subject's brain tissue may be determined. An analysis of the temporal fluctuations in (e.g. decay of) DTOF values over time may provide an indication of one or more dynamic properties of the subject's brain tissue. That is, the decay analysis may be used to identify that one or more properties within the subject's brain tissue are changing, as well as optionally identifying the rate at which these properties are changing (e.g. using the decay rate). The order of the decay of DTOF (e.g. decaying with t, t², etc.) may be used to determine one or more properties of the type of motion (e.g. diffusion or flow).

The controller 40 may store data which correlates time of flight for a sample light photon to an indication of average path trajectory for that photon. This may include an indication of the depth of the penetration into the subject's brain tissue for that photon, and/or an indication of the region(s) of the subject's brain tissue through which that photon travelled from light source 20 to light detector 30. The controller 40 may be configured to process the DTOF data by dividing this data up into selected time of flight bins. Within each TOF bin, the data may provide depth-resolved evolution data for the subject's brain tissue. That is, as the TOF may be associated with certain penetration depths or regions, each TOF bin may contain data showing properties associated with a certain penetration depth or region. The evolution of data within each TOF bin may therefore provide an indication of how one or more properties of the subject's brain tissue are evolving. For example, where the evolution suggests a change in movement (e.g. a flow of blood), that movement may be identified, as may the region in which that movement is occurring. For this, a TOF-resolved decay slope may be used to identify how the curve is decaying over time for specific TOFs (e.g. for specific penetration depths/regions).

In other words, the iNIRS system 10 is configured to perform an autocorrelation in which DTOFs for successive wavelength sweeps are combined to assess fluctuations in the light field at the light detector 30 over time. The fluctuations may be identified due to relevant decay in DTOFs over time. The fluctuations may also be depth-resolved, by identifying the relevant TOFs at which those fluctuations are occurring (and thus the relevant penetration depths/regions).

The controller 40 may be configured to process the data received from the light detector(s) 30 to provide time of flight information with depth-resolved autocorrelations for the subject's brain tissue. The controller 40 may be configured to use this information to obtain an indication of a plurality of different properties of the subject's brain tissue, such as intracranial pressure (‘ICP’), blood flow index, artery elasticity, cerebral metabolic rate of oxygen consumption (CMRO₂) etc. These properties of the subject's brain tissue may be used for a plurality of different forms of neuroimaging and analysis, such as in a brain-computer interface, for imaging regions of the brain to identify potential localised injury or strokes, and/or to monitor neuro responses to substances, such as drugs.

### Blood Oxygenation Determination

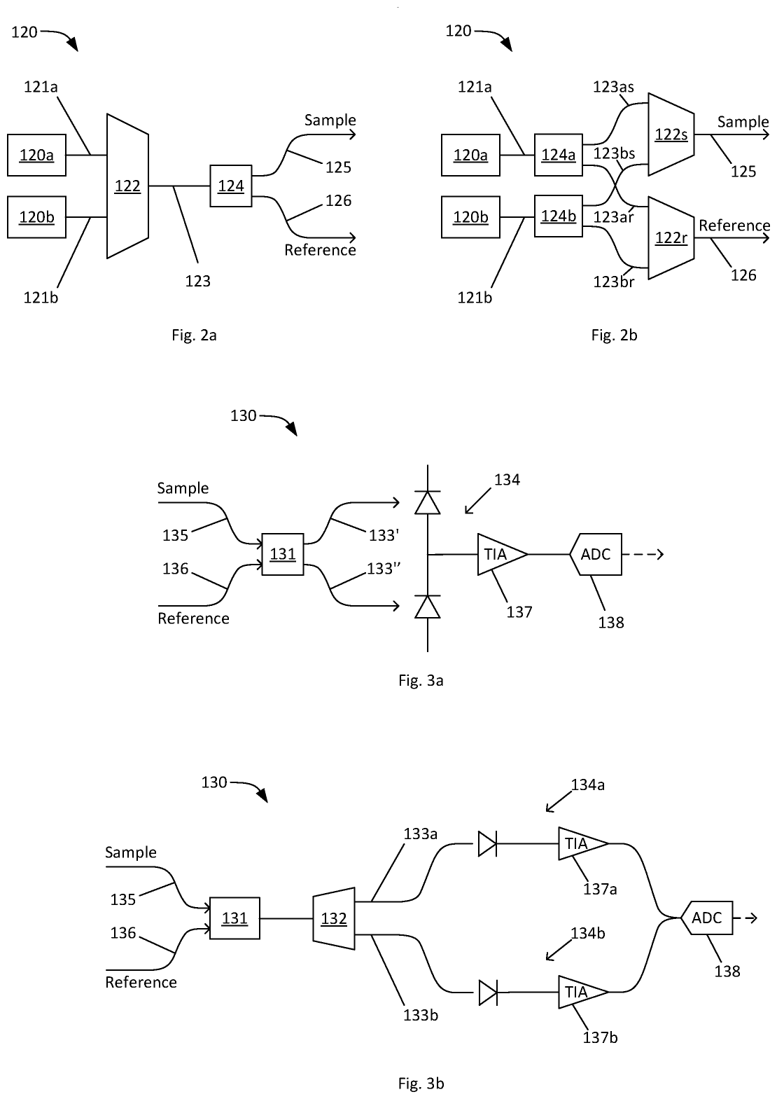

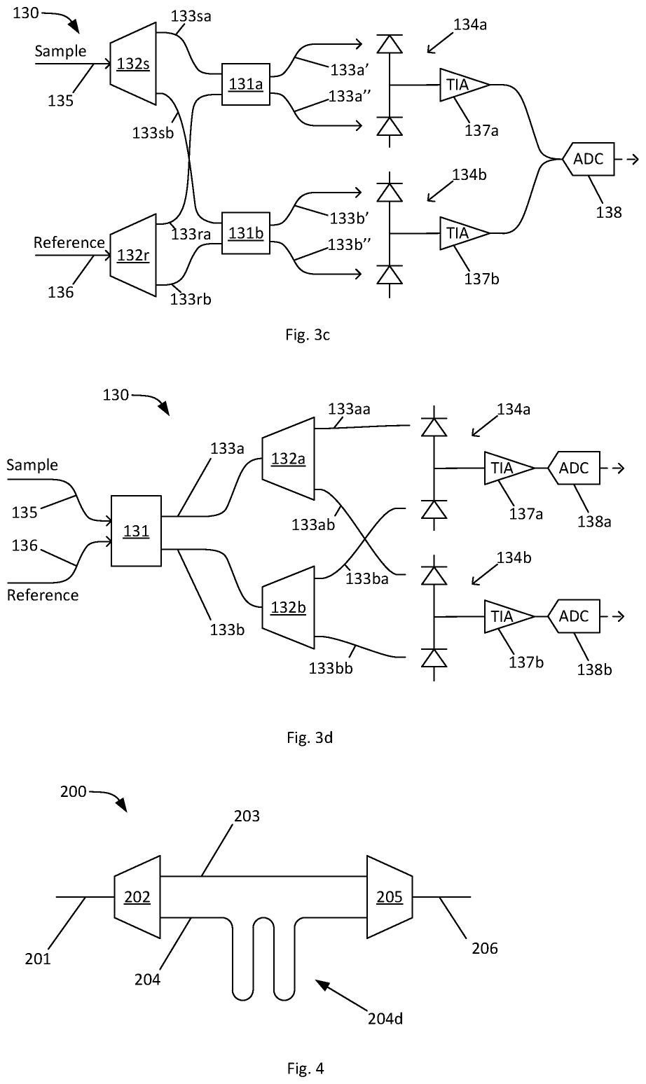

Reference will now be made to FIGS. 2a to 3d, which show components for an iNIRS system.

FIGS. 2a and 2b show arrangements for outputting sample and reference light (i.e. with sample light to be delivered towards a subject's brain tissue, and reference light to be delivered directly to the detector along one or more reference channels). The light source arrangements of FIGS. 2a and 2b are arranged for outputting sample and reference light from two light sources, with one light source emitting light at wavelengths above an oximetry isosbestic wavelength, and the other light source emitting light at wavelengths below the oximetry isosbestic wavelength. In particular, the light source arrangements of FIGS. 2a and 2b are configured to deliver light from each light source onto the same single common light channel as that light travels from its light source towards the subject's scalp. Some of the light from each light source is also delivered to a reference channel for directly connecting the light source to the light detector(s).

FIGS. 3a to 3d show arrangements for receiving sample and reference light (i.e. with sample light arriving from the subject's brain tissue, and reference light arriving directly from the light source). The light detection arrangements of FIGS. 3a to 3d are arranged for receiving sample and reference light from two light sources, with the light from one light source at wavelengths above an oximetry isosbestic wavelength, and with the light from the other light source at wavelengths below the oximetry isosbestic wavelength.

The light source arrangements in FIGS. 2a and 2b, and/or the light detection arrangements in FIGS. 3a to 3d may be provided in the iNIRS system shown in FIG. 1 (and as described above). In particular, embodiments of the present disclosure may provide an iNIRS system having the light generating arrangement from FIGS. 2a and/or 2b, as well as the light receiving arrangement from any of FIGS. 3a to 3d. As such, embodiments may provide an iNIRS system designed to emit and detect light at wavelengths either side of the oximetry isosbestic wavelength for determining blood oxygenation concentrations for the subject's brain tissue.

FIG. 2a shows a light emitting arrangement 120. The light emitting arrangement 120 includes a first light source 120a (above oximetry isosbestic wavelength) and a second light source 120b (below oximetry isosbestic wavelength). The light emitting arrangement 120 includes a common light combiner 122, a common light channel 123, and a common light splitter 124. As with the iNIRS system described above, the light emitting arrangement 120 includes a common sample delivery channel 125 and a common reference delivery channel 126.

The common light combiner 122 couples the two light sources to the common light channel 123. The first light source 120a may be coupled to the common light combiner 122 via a first coupling channel 121a, and the second light source 120b may be coupled to the common light combiner 122 via a second coupling channel 121b. The common light channel 123 couples the common light combiner 122 to the common light splitter 124. The common light channel 123 may be a single optical channel. The common light channel 123 may extend from the common light combiner 122 to the common light splitter 124. The common light splitter 124 couples the single common light channel 123 to two separate channels: the common sample delivery channel 125 and the common reference delivery channel 126. As set out above, the common sample delivery channel 125 may be coupled to the subject's scalp in one or more regions, e.g. via one or more sample delivery probes. Similarly, the common reference delivery channel 126 may be coupled to one or more of the light detectors, e.g. via one or more reference receiving channels.

The light emitting arrangement 120 is configured to deliver both: (i) sample light to the subject's brain tissue (e.g. through the common sample delivery channel 125), and (ii) reference light to one or more light detectors (e.g. through the common reference delivery channel 126). The light emitting arrangement 120 is configured so that light from each of the two light sources will travel along the same optical channel. For example, sample light from the first light source 120a and sample light from the second light source 120b will each travel along at least one common light channel. In the example shown in FIG. 2a, sample light from the two light sources will travel along the same common light channel 123, as well as the same common sample delivery channel 125. For example, reference light from the first light source 120a and reference light from the second light source 120b will each travel along at least one common light channel. In the example shown in FIG. 2a, reference light from the two light sources will travel along the same common light channel 123, as well as the same common reference delivery channel 126.

A similar light emitting arrangement is also shown in FIG. 2b.

FIG. 2b shows a light emitting arrangement 120. As with FIG. 2a, the light emitting arrangement 120 includes a first light source 120a and a second light source 120b. Similarly, the first light source 120a may be coupled to a first coupling channel 121a, and the second light source 120b may be coupled to a second coupling channel 121b.

In FIG. 2b, light from each light source first passes through a light splitter before light from the two light sources are combined onto a same light channel. For this, the light emitting arrangement 120 of FIG. 2b includes a first light splitter 124a and a second light splitter 124b. The first light source 120a is coupled to the first light splitter 124a (e.g. via the first coupling channel 121a), and the second light source 120b is coupled to the second light splitter 124b (e.g. via the second coupling channel 121b). The light emitting arrangement 120 of FIG. 2b includes two common light combiners: a sample common light combiner 122s and a reference common light combiner 122r. The sample common light combiner 122s is coupled to each of the first light splitter 124a and the second light splitter 124b, and also to a common sample delivery channel 125. The reference common light combiner 122r is coupled to each of the first light splitter 124a and the second light splitter 124b, and also to a common reference delivery channel 126. The common sample delivery channel 125 may be coupled to the subject's scalp in one or more regions, e.g. via one or more sample delivery probes. Similarly, the common reference delivery channel 126 may be coupled to one or more of the light detectors, e.g. via one or more reference receiving channels.

The first light splitter 124a couples the first light source 120a to each of two separate optical channels: a first sample delivery channel 123as and a first reference delivery channel 123ar. The second light splitter 124b couples the second light source 120b to each of two separate optical channels: a second sample delivery channel 123bs and a second reference delivery channel 123br. The first sample delivery channel 123as couples the first light splitter 124a (and thus the first light source 120a) to the sample common light combiner 122s. The first reference delivery channel 123ar couples the first light splitter 124a (and thus the first light source 120a) to the reference common light combiner 122r. The second sample delivery channel 123bs couples the second light splitter 124b (and thus the second light source 120b) to the sample common light combiner 122s. The second reference delivery channel 123br couples the second light splitter 124b (and thus the second light source 120b) to the reference common light combiner 122r. The sample common light combiner 122s couples both of the first sample delivery channel 123as and the second sample delivery channel 123bs to the common sample delivery channel 125. The reference common light combiner 122r couples both of the first reference delivery channel 123ar and the second reference delivery channel 123br to the common reference delivery channel 126.

In the light emitting arrangement 120 of FIG. 2b, there are two common light channels, where light from either light source will travel along those common light channels. These are the common sample delivery channel 125 and the common reference delivery channel 126. Each of these common light delivery channels is coupled to a common light combiner to receive light from one or both of the two light sources. One or both of these common light channels may be a single optical channel. For example, the common sample delivery channel 125 may be a single optical channel which extends from the sample common light combiner 122s towards the subject's brain tissue. For example, the common reference delivery channel 126 may be a single optical channel which extends from the reference common light combiner 122r towards one or more optical detectors.

The light emitting arrangement 120 is configured to deliver both: (i) sample light to the subject's brain tissue (e.g. through the common sample delivery channel 125), and (ii) reference light to one or more light detectors (e.g. through the common reference delivery channel 126). The light emitting arrangement 120 is configured so that light from each of the two light sources will travel along the same optical channel. For example, sample light from the first light source 120a and sample light from the second light source 120b will each travel along at least one common light channel. In the example shown in FIG. 2b, sample light from the two light sources will travel along the same common sample delivery channel 125. For example, reference light from the first light source 120a and reference light from the second light source 120b will each travel along at least one common light channel. In the example shown in FIG. 2b, reference light from the two light sources will travel along the same common reference delivery channel 126.

The functionality of the two light emitting arrangements 120 will be described below together.

The light emitting arrangement 120 in each of FIGS. 2a and 2b is configured to deliver sample light towards the subject's brain tissue from two separate light sources, where sample light from each light source will travel along at least one common optical channel as it travels from either light source towards the subject's scalp. Similarly, the light emitting arrangement 120 in each of FIGS. 2a and 2b is configured to deliver reference light towards one or more detectors from two separate light sources, where reference light from each light source will travel along at least one common optical channel as it travels from either light source towards the one or more detectors.

For each light emitting arrangement 120, each of the first light source 120a and the second light source 120b is similar to the light source described above in relation to FIG. 1. That is, each light source is configured to provide wavelength swept emission of light. These details for the functionality of first light source 120a and the second light source 120b will not be described again, as they have already been described above. The first and second light sources differ in the wavelengths of light they emit. The primary wavelengths for the first light source 120a (e.g. a first laser) and the second light source 120b (e.g. a second laser) will be different.

The first light source 120a is arranged to emit light at wavelengths above an oximetry isosbestic wavelength. The second light source 120b is arranged to emit light at wavelengths below an oximetry isosbestic wavelength. An oximetry isosbestic wavelength is a wavelength at which absorption properties of oxygenated and de-oxygenated haemoglobin are the same (e.g. where they have the same absorption coefficients). At wavelengths above and below the oximetry isosbestic wavelength, absorption coefficients for oxygenated and de-oxygenated haemoglobin will be different (except at any other isosbestic wavelengths). In other words, the oximetry isosbestic wavelength is the wavelength at which the values of the wavelength-dependant absorption coefficients for oxygenated and de-oxygenated haemoglobin swap. For oximetry of haemoglobin, there are two relevant oximetry isosbestic wavelengths: one at approximately 590 nm and one at approximately 805 nm. For example, the oximetry isosbestic wavelength used in the present disclosure may be at approximately 805 nm, such as at roughly 808 nm.

The first light source 120a is configured to provide wavelength-swept emission through a plurality of wavelengths greater than the oximetry isosbestic wavelength (e.g. greater than 808 nm). As set out above in relation to FIG. 1, the first light source 120a may be a laser configured to emit light at a selected wavelength, and the light emitting arrangement 120 may also include a light source modifier configured to vary the wavelength of light emitted around this selected wavelength (e.g. to provide wavelength sweeping through wavelengths around the selected wavelength at which that laser emits light). For example, the first light source 120a may be configured to emit light at a selected wavelength of approximately 830, 850 or 900 nm. Preferably, the first light source 120a may be configured to emit light at a wavelength of 852 nm. The light emitting arrangement 120 is configured to provide wavelength sweeping for the first light source 120a through a plurality of wavelengths at and around 852 nm.

The second light source 120b is configured to provide wavelength-swept emission through a plurality of wavelengths below than the oximetry isosbestic wavelength (e.g. below 808 nm). For example, the second light source 120b may comprise a laser configured to emit light at a selected wavelength of approximately 690, 760 or 785 nm. Preferably, the second light source 120b may be configured to emit light at a wavelength of 780 nm. The light emitting arrangement 120 is configured to provide wavelength sweeping for the second light source 120b through a plurality of wavelengths at and around 780 nm. For example, the first and second light sources may be utilise wavelengths in the range of 760 nm to 880 nm, with one wavelength above the oximetry isosbestic wavelength, and the other beneath it. For example, the first light source 120a may emit light at approximately 830 nm or 850 nm (e.g. at 830 nm or 850 nm), and the second light source 120b may emit light at approximately 780 nm (e.g. at 780 nm).

As set out above, the light emitting arrangement 120 is arranged to provide wavelength-swept emission at wavelengths above the oximetry isosbestic wavelength and at wavelengths below the oximetry isosbestic wavelength. The light emitting arrangement 120 is also arranged to couple light from both of the light sources onto one or more common optical channels. Each optical channel may comprise a fibre optic cable.

As set out above in relation to FIG. 1, light combiners of the present disclosure are arranged to combine two separate input streams of light onto a single optical channel. For example, each light combiner may be configured to receive input light from two separate channels, e.g. two separate optical fibre cables, and to combine those two streams of input light onto a single optical channel (as an output). The light splitters are arranged to separate one input stream from a single optical channel into two separate optical channels. For example, each light splitter may be configured to receive input light from a single channel, e.g. one common optical fibre cable, and to split that stream of input light onto two separate optical channels (e.g. as two outputs). Each light combiner may be a wavelength combiner, e.g. a wavelength division multiplexer (WDM). Each light separator may be a wavelength separator in reverse. For example, a wavelength combiner/separator (e.g. a WDM) may comprise one or more gratings and/or prisms arranged to split and/or combine light, as relevant. Additionally, or alternatively, combiners and/or separators could be implemented with discrete components (e.g. in free space). Fibre couplers could be used instead of, or in addition to, a WDM, and/or a WDM may be used for light combining/splitting without being used for multiplexing and/or demultiplexing.

The light emitting arrangement 120 is operable to provide two active wavelength outputs from the same device. At least one common channel is used for delivering the light from either or both light sources to be output. The light emitting arrangement 120 may be configured to output the light from both light sources simultaneously. In which case, light from both light sources may travel simultaneously along the same at least one common optical channel. The light emitting arrangement 120 may be configured to temporally interleave the operation of the two light sources so that light is output from one light source at a time. In which case, while light from both light sources will travel along the same common optical channel, only light from one light source may travel along that channel at any one time.

The light emitting arrangement 120 may be arranged so that the reference light from the first light source 120a travels a different distance to the reference light from the second light source 120b (when travelling from the respective light source to each detector). For example, the reference path for one of the light sources to the light detectors may include a surplus amount of optical channel, so that the reference light from that light source will take longer to travel to the detector than the reference light from the other light source. The length of optical channel along which light from the two light sources travel for sample light to be delivered towards the subject's brain tissue may be approximately the same (e.g. sample light from either light source may travel the same distance towards the subject's brain tissue).

For example, in FIG. 2b, the distance from the first light source 120a to the common sample delivery channel 125 may be the same, or approximately the same, as the distance from the second light source 120b to the common sample delivery channel 125. That is, the first sample delivery channel 123as and the second sample delivery channel 123bs may be of the same length (and/or where first and second coupling channels are used, the combined length of the first coupling channel 121a and the first sample delivery channel 123as may be the same as the combined length of the second coupling channel 121b and the second sample delivery channel 123bs). However, the distance from the first light source 120a to the common reference delivery channel 126 may be different to the distance from the second light source 120b to the common reference delivery channel 126. That is, the first reference delivery channel 123ar and the second reference delivery channel 123br may be of a different length (and/or where first and second coupling channels are used, the combined length of the first coupling channel 121a and the first reference delivery channel 123ar may be different to the combined length of the second coupling channel 121b and the second reference delivery channel 123br). One of the sample or reference channels may be arranged to provide a difference in relative delay for the light to be combined.

The difference in distance for the relevant reference path length may be selected so that the resulting beat frequencies between sample light and reference light from one of the light sources are different to the resulting beat frequencies between sample light and reference light from the other of the light sources. In other words, by virtue of the reference light for one of the light sources travelling along a longer path, the difference in time of flight between reference and sample light from that light source will be different to that from the other light source. Typically, the longer reference path will lead to smaller beat frequencies, as the difference in time of flight between sample and reference light, as received at the detector, will be less. A difference in optical path length for reference light from the two light sources may be selected so that there is no, or minimal, spectral overlap between: (i) beat frequencies for sample and reference light from the first light source 120a, and (ii) beat frequencies for sample and reference light from the second light source 120b. As described in more detail below for the detectors, this approach may be used so that light from both light sources may be emitted and detected simultaneously, while enabling the detected signals associated with the two different light sources to be separately identifiable.

Some example light detecting arrangements will now be described with reference to FIGS. 3a to 3d, and then operation of these detecting arrangements will be described in combination with corresponding operation of the different light emitting arrangements 120.

FIG. 3a shows a light detecting arrangement 130. The light detecting arrangement 130 has a sample receiving channel 135 and a reference receiving channel 136. The light detecting arrangement 130 has a detection light combiner and splitter 131, a first detection channel 133′, a second detection channel 133″. The light detecting arrangement 130 includes at least one element for converting a received optical signal into an electrical signal, e.g. a photodetector, such as a photodiode. In the example of FIG. 3a, this is a balanced photodetector 134. Also shown in FIG. 3a is a transimpedance amplifier 137 and an analogue to digital converter 138.

The sample receiving channel 135 is arranged to be coupled to the subject's scalp, e.g. via one or more sample receiving probes. The reference receiving channel 136 is arranged to be coupled to both the first and second light sources, e.g. the reference receiving channel 136 may be coupled to one of the reference delivery channels in FIGS. 2a and 2b. Both the sample receiving channel 135 and the reference receiving channel 136 are coupled to the light combiner and splitter. The light combiner and splitter is coupled to both the first detection channel 133′ and the second detection channel 133″. The two detection channels are coupled to the balanced photodetector 134. An output of the balanced photodetector 134 is coupled to the analogue to digital converter 138 via the transimpedance amplifier 137.

The light detecting arrangement 130 is configured to combine the sample and reference light to provide a combined signal, and to analyse this combined signal to determine properties of the subject's brain tissue. In the example of FIG. 3a, the detection light combiner and splitter 131 is configured to combine sample light from the sample receiving channel 135 with reference light from the reference receiving channel 136 to provide a combined light signal, and to split the combined light signal into two separate optical channels: the first detection channel 133′ and the second detection channel 133″. The light detecting arrangement 130 is arranged to provide this light in the two detection channels as the input to the balanced photodetector 134. The balanced photodetector 134, transimpedance amplifier 137 and analogue to digital converter 138 are configured to process this light input as disclosed above.

A similar light detecting arrangement is shown in FIG. 3b.

FIG. 3b shows a light detecting arrangement 130. As with FIG. 3a, this includes a sample receiving channel 135, and a reference receiving channel 136. The light detecting arrangement 130 also includes a detection light combiner 131 and a wavelength demultiplexer 132. The wavelength demultiplexer 132 is arranged to demultiplex light associated with the first light source from light associated with the second light source. The wavelength demultiplexer 132 demultiplexes light into two channels: a first light source detection channel 133a, and a second light source detection channel 133b (i.e. one optical channel for light from each light source). Each of the light source detection channels has an associated photodetector (first photodetector 134a and second photodetector 134b). These may be single photodetectors (e.g. single-ended, not balanced photodetectors). Each photodetector may have an associated TIA (first TIA 137a and second TIA 137b), and the arrangement 130 also includes at least one ADC (ADC 138).

The detection light combiner 131 is coupled to both the sample light receiving channel and the reference light receiving channel. The detection light combiner 131 is coupled to the wavelength demultiplexer 132 (e.g. via an optical channel). The wavelength demultiplexer 132 is coupled to each of the first light source detection channel 133a and the second light source detection channel 133b. Each of the light source detection channels couples the wavelength demultiplexer 132 to a respective photodiode (first photodiode 134a or second photodiode 134b).

The light detecting arrangement 130 is configured to combine the sample and reference light to provide a combined signal, and to analyse this combined signal to determine properties of the subject's brain tissue. In the example of FIG. 3b, the detection light combiner 131 is configured to combine sample light from the sample receiving channel 135 with reference light from the reference receiving channel 136 to provide a combined light signal. The wavelength demultiplexer 132 is configured to split this combined light signal into two separate optical channels so that light associated with the first light source 120a is provided to a different channel (the first light source detection channel 133a) to the light associated with the second light source 120b (the second light source detection channel 133b). The light signals in these two channels will be processed separately, e.g. to provide two digital outputs, one for light from each light source.

FIG. 3c shows a light detecting arrangement 130. As with FIGS. 3a and 3b, this includes a sample receiving channel 135, and a reference receiving channel 136. Each channel is connected to a corresponding wavelength demultiplexer (sample light wavelength demultiplexer 132s and reference light wavelength demultiplexer 132r). The light detecting arrangement 130 also includes a first light source combiner and splitter 131a and a second light source combiner and splitter 131b.

The sample light wavelength demultiplexer 132s has an input coupled to the sample receiving channel 135 and two outputs: one coupled to the first light source combiner and splitter 131a (via a first sample delivery channel 133sa) and one coupled to the second light source combiner and splitter 131b (via a second sample delivery channel 133sb). The reference light wavelength demultiplexer 132r has an input coupled to the reference receiving channel 136 and two outputs: one coupled to the first light source combiner and splitter 131a (via a first reference delivery channel 133ra) and one coupled to the second light source combiner and splitter 131b (via a second reference delivery channel 133rb). The first light source combiner and splitter 131a is coupled to a balanced photodetector 134a (via channels 133a′ and 133a″ respectively). The second light source combiner and splitter 131b is coupled to a balanced photodetector 134b (via channels 133b′ and 133b″ respectively). Each balanced photodetector is coupled to ADC 138 via a respective TIA (first TIA 137a and second TIA 137b).

The light detecting arrangement 130 is configured to demultiplex the light associated with the first light source from the light associated with the second light source for each of the sample and reference light received via the sample and reference channels 135 and 136 respectively. Each of the first and second light source combiner and splitters 131a and 131b is configured to combine and split its respective sample and reference light to be detected by its associated balanced photodetector. The light signals in these two channels will be processed separately, e.g. to provide two digital outputs, one for light from each light source.

FIG. 3d shows a light detecting arrangement 130. The arrangement 130 includes a sample receiving channel 135 and a reference receiving channel 136. The arrangement also includes a light combiner and splitter 131, a first wavelength demultiplexer 132a and a second wavelength demultiplexer 132b.

The light combiner and splitter 131 is coupled to each of the sample delivery channel 135 (to receive sample light from both light sources) and the reference delivery channel 136 (to receive reference light from both light sources). The light splitter and combiner 131 is coupled to: the first wavelength division demultiplexer 132a (via channel 133a), and the second wavelength division demultiplexer 132b (via channel 133b). The first wavelength division demultiplexer 132a has two outputs: a first is coupled to a first photodetector of a first balanced photodetector 134a (via channel 133aa), and a second is coupled to a first photodetector of a second balanced photodetector 134b (via channel 133ab). The second wavelength division demultiplexer 132b has two outputs: a first is coupled to a second photodetector of the first balanced photodetector 134a (via channel 133ba), and a second is coupled to a second photodetector of the second balanced photodetector 134b (via channel 133bb). The first balanced photodetector 134a is coupled to a respective first TIA 137a and an ADC channel (ADC 138a). The second balanced photodetector 134b is coupled to a respective second TIA 137b and an ADC channel (ADC 138b).

The arrangement 130 is configured to provide light signals associated with the first light source to a first digitising stream (via first balanced photodetector 134a) and light signals associated with the second light source to a second digitising stream (via second balanced photodetector 134b).

In FIGS. 3a to 3d examples are shown with one or two ADC channels. It is to be appreciated that, in the event that an optical delay is introduced to avoid spectral overlap associated with beat frequencies, then both combined light signals could be digitised simultaneously using a single channel of an ADC. In the event that no such optical delay is introduced, then separate ADC channels could be used for providing simultaneous digitising for light from the first and second light sources.

The functionality of the light detecting arrangements 130 will be described below together.

Each light detecting arrangement 130 is configured to operate in the same manner as the detector described above in relation to FIG. 1. That is, each light detecting arrangement 130 is configured to combine sample and reference light to provide a combined light signal comprising one or more components at a beat frequency between the sample light and reference light. Here, the sample light will contain light from the first light source 120a (e.g. at wavelengths above the oximetry isosbestic wavelength) which has travelled through the subject's brain tissue, and/or light from the second light source 120b (e.g. at wavelengths below the oximetry isosbestic wavelength) which has travelled through the subject's brain tissue. Similarly, the reference light will contain light from the first light source 120a which has travelled along one or more reference channels, and/or light from the second light source 120b which has travelled along one or more reference channels. As such, each light detecting arrangement 130 will be receiving light associated with two different light sources (and thus also two different wavelength ranges-one above, and one below, the oximetry isosbestic wavelength).

The iNIRS system may include a controller configured to process combined light signals associated with each light source in the same manner as disclosed above in relation to FIG. 1. That is, absorption and/or scattering coefficients may be determined, as may depth-resolved information for the subject's brain tissue (e.g. based on time-of-flight distributions for photons travelling through the subject's brain tissue), as well as other properties such as an indication of blood flow etc.

In particular, for light from each light source, the controller may be configured to determine absorption characteristics, e.g. absorption coefficients, associated with the light being emitted from that light source. The controller may determine absorption coefficients for the two different wavelength ranges (e.g. for the greater wavelength light from the first light source 120a and the smaller wavelength light from the second light source 120b). The controller may be configured to compare the absorption coefficients for the two wavelength ranges to determine an indication of relative concentrations of oxygenated and de-oxygenated haemoglobin. In other words, the controller may be configured to determine a value for blood oxygen concentration for the subject's brain tissue). As described above, the controller may be configured to provide depth-resolved information, e.g. depth-resolved blood oxygen concentration levels, by analysing data associated with different photon times of flight through the subject's brain tissue. The controller may be configured to determine an indication of an absolute amount of oxygen in the blood based on the oxygen concentration.

The light emitting arrangements 120 and light detecting arrangements 130 may be operated to obtain an indication of blood oxygen concentration for the subject's brain tissue by emitting and detecting light in two separate wavelength ranges (one either side of the oximetry isosbestic wavelength). This functionality may be provided in a single device, e.g. rather than requiring separate components to measure different properties. To reduce the requirements for the device, the iNIRS system may be configured to control operation of the light emitting arrangement 120 and/or light detecting arrangement 130 so that measurements for blood oxygenation concentration may be obtained using a single detector (e.g. with a single interferometer, a single sample receiving channel 135, and a single reference receiving channel 136). Similarly, each light emitting arrangement 120 may be coupled to a plurality of different light detecting arrangements 130, which could still detect light from that light source. For this:
1. The light emitting arrangement 120 may be configured to: (i) time multiplex the sample and reference light, (ii) wavelength multiplex the sample and reference light, and/or (iii) provide a different optical path length for reference light from the two light sources.
2. The light detecting arrangement 130 may be configured to wavelength demultiplex the received sample and reference light (e.g. so that the output from each photodetector is representative of only one of the light sources).
3. The controller (signal processing circuitry in hardware/software) may be configured to: (i) attribute obtained data with relevant light source based on time multiplexing scheme, (ii) attribute data from each photodetector to a corresponding light source, and/or (iii) attribute data in different frequency bands to corresponding light source.

For example, the iNIRS system may be configured to provide time-domain multiplexed detection. For this, operation of the two light sources is temporally interleaved so that one light source operates at a time (e.g. for a selected number of wavelength sweeps). In this case, the output from the detector will either be indicative of one of the first light source 120a or the second light source 120b, depending on the time. The controller may store an indication of the temporal modulation pattern of the light emitting arrangement 120 (such as an indication of an emitting start time, a sweep rate, and/or a number of sweeps per light source before switching the active light source). The controller may be configured to process output data from the light detecting arrangement 130 so that this data is filtered according to the operation timings, such that data can be isolated for each of the two light sources.

As another example, the iNIRS system may be configured to provide wavelength-domain multiplexed detection. For this, wavelength multiplexing is performed for the sample light from the two light sources, with simple light from both light sources being emitted simultaneously. The light detecting arrangement 130 may include a sample light splitter, such as a wavelength demultiplexer, configured to de-multiplex the received light (both sample and reference) into separate channels based on the wavelength multiplexed scheme as applied by a wavelength multiplexer of the light emitting arrangement 120. The light detecting arrangement 130 may therefore split the received light into two separate signal processing channels: one for light from each light source, so that received light from each light source may be processed independently. The two signal processing channels may thus provide independent outputs: one for each light source, and the controller may process the output data for the two signal processing channels independently.

As a third example, the iNIRS system may be configured to provide an optical path length offset between the two light sources and the detector. The beat frequencies associated with one of the light sources may therefore be in a different range to the beat frequencies associated with the other light source. The light detecting arrangement 130 may be configured to obtain spectral data formed of two separate components (each component associated with a respective light source) in separable frequency ranges. The signal processing circuitry and/or the controller may be configured to process this received light signal/data output from the ADC to associate the data with the two separable frequency ranges with the corresponding light source.

One example of such a delay arrangement is shown in FIG. 4. This delay arrangement is configured for use in a common optical path (i.e. where light may be simultaneously transmitted from both the first and second light sources). The common optical path could be in a sample or reference arm (or in a common channel before splitting into separate reference/sample channels).

FIG. 4 shows a delay arrangement 200 for including in a common optical channel. The delay arrangement 200 has an input channel 201 and an output channel 206. The input channel 201 and output channel 206 may be the remaining portions of the common optical channel in which the delay arrangement 200 is provided. The delay arrangement 200 also includes a wavelength demultiplexer 202, a first channel 203, a second channel 204, and a wavelength multiplexer 205. The wavelength demultiplexer has an input coupled to the input channel 201 and two outputs: one coupled to the first channel 203, and one coupled to the second channel 204. The wavelength multiplexer has two inputs: one coupled to the first channel 203, and one coupled to the second channel 204, and an output coupled to the output channel 206. The second channel 204 includes a delay portion 204d. The delay portion includes an additional length of channel, such that the optical path length along the first channel 203 (from demultiplexer 202 to multiplexer 205) is shorter then the optical path length along the second channel 204 (from demultiplexer 202 to multiplexer 205).

The demultiplexer 202 is arranged to wavelength demultiplex the light it receives into the two separate channels. For example, the demultiplexer 202 may be configured to demultiplex light from the first light source onto the first channel 203 and light from the second light source onto the second channel 204. The multiplexer 205 is configured to multiplex light from the first channel 203 and the second channel 204 onto a common channel (the output channel 206). The delay portion 204d has a selected length such that resulting beat frequencies generated by combining sample and reference light from the first and second light sources have minimal or no spectral overlap with each other. For example, where light from the second light source travels through the delay portion 204d, this may result in the time of flight difference for sample and reference light from the second light source being substantially different to that for sample and reference light from the first light source (e.g. the delay could be smaller or larger).

For FIGS. 3a to 3d, the light detecting arrangement 130 may be configured to provide an output (e.g. a digital output from the ADC), which contains at least one of: (i) data which alternates between being indicative of the first light source 120a or being indicative of the second light source 120b (e.g. where that data may be separated and associated with the relevant light source using information about the timing for operation of the two light sources), (ii) concurrent data for both light sources, but from separate signal processing flows (e.g. where data output for each signal processing flow is indicative of one of the light sources), and/or (iii) data which contains concurrent data for both light sources, but which can be separated into two separate data streams (e.g. based on beat frequencies).

Three examples of operating an iNIRS system will now be described with reference to the light emitting arrangements 120 and light detecting arrangements 130 shown in FIGS. 2a to 3d.

In a first example, the light emitting arrangement 120 of FIG. 2a may be used in combination with the light detecting arrangement 130 of FIG. 3a.

The controller controls operation of the first light source 120a and the second light source 120b so that their operation is temporally interleaved. For a first time period, the first light source 120a will provide wavelength swept emission of light. This light is provided from the first light source 120a to the common light channel 123 (via the common light combiner 122), and this light is split by the common light splitter 124 onto the common sample delivery channel 125 and the common reference delivery channel 126. Reference light travels along the common reference delivery channel 126 to where it is received at the reference receiving channel 136. Sample light travels along the common sample delivery channel 125 and into the subject's brain tissue, where it scatters and is received at the sample receiving channel 135. The light detecting arrangement 130 combines the sample and reference light and processes this combined light. The controller processes obtained data for this first time period to determine an indication of the absorption coefficients for the relevant wavelength(s) during the first time period. This process is then repeated for a second time period, except it is the second light source 120b which is emitting light. This time, the controller processes obtained data for this second time period to determine an indication of the absorption coefficients for the relevant wavelength(s) during the second time period. The controller compares any absorption coefficients associated with the first time period with any absorption coefficients associated with the second time period to determine the ratio of oxygenated to de-oxygenated haemoglobin in the subject's blood.

In a second example, the light emitting arrangement 120 of FIG. 2b may be used in combination with the light detecting arrangement 130 of FIG. 3a.

The first light source 120a and the second light source 120b emit light simultaneously. Light from each light source travels to both the common sample delivery channel 125, and the common reference delivery channel 126. The light from one of the light sources travels further to get to the common reference delivery channel 126 than for the other light source. The resulting wavelength differences between received sample light and reference light from one light source will be different to those from the other light source (due to the increased reference light optical path length). The light detecting arrangement 130 may obtain an indication of absorption data for both light sources simultaneously-with one light source's data being at greater beat frequencies than for the other. In other words, the resulting data may contain two subsets: one associated with light from each light source. The controller processes this data to extract information for each light source. The controller then determines absorption coefficients for the two light sources (for obtaining blood oxygen concentration data).

In a third example, the light emitting arrangement 120 of FIG. 2b may be used in combination with the light detecting arrangement 130 of FIG. 3c or 3d.

As with the second example, the first light source 120a and the second light source 120b operate simultaneously, but the reference optical path lengths could be the same. Light from each light source is wavelength multiplexed onto each of the common sample delivery channel 125 and the common reference delivery channel 126. Light from both light sources may therefore travel along these optical channels, but this light may ultimately be separated back out into its two component parts using a corresponding wavelength demultiplexing scheme. At the light detecting arrangement 130, sample and reference light is wavelength de-multiplexed to split the combined light signal out into two constituent parts: one for light associated with each of the two light sources (e.g. as in FIG. 3c). Alternatively, at the light detecting arrangement 130, sample and reference light is combined, and this combined light signal is then wavelength de-multiplexed to split the combined light signal out into two constituent parts: one for light associated with each of the two light sources (e.g. as in FIG. 3d). Light associated with each light source is processed individually (as with the first example), and the controller may determine absorption coefficients/blood oxygenation data based on the two separate outputs (associated with each light source).

Although not shown in the Figs., light emitting arrangements of the present disclosure may utilise one or more optical amplifiers. For example, a small signal amplifier may be used. This may be based on a semiconductor gain chip (e.g. but without the cavity to make it a laser). The optical amplifier may be provided in a sample arm and only some of the light from the light source is provided to the amplifier (e.g. 10-20%) for amplification, where the rest is to be provided to the reference channel(s)/detector(s). As another example, a high-power amplifier may be used, such as one based on a tapered amplifier design. The optical amplifier may be arranged to provide optical amplification of the light emitted from the first and/or second light sources (e.g. for sample and/or reference light).

Although not shown, the light emitting arrangement of the present disclosure may include a plurality of light delivery channels for delivery light from the first and second light source 120b to the subject's scalp. For this, the light emitting arrangement may include an optical switch and a plurality of light delivery channels which are coupled to the common sample delivery channel 125. The optical switch may be operable to direct light from the common sample delivery channel 125 (e.g. from the first and/or second light source) onto each of the different sample delivery channels. The different sample delivery channels may be coupled to the subject's scalp at a plurality of different locations (e.g. used sample probes). The optical switch may select one light delivery channel at a time, e.g. it may switch through a selected pattern for when light is delivered to the subject's scalp through each of the different light delivery channels.

For iNIRS systems of the present disclosure, there may be a plurality of light sources and/or optical detectors. The iNIRS system may comprise a plurality of light detecting arrangements 130 coupled to the same light emitting arrangement. Each of the optical detectors may be coupled to the same common reference delivery channel 126 of the light emitting arrangement (e.g. as shown in FIG. 1) to receive reference light from the first and second light sources (e.g. via that same common reference delivery channel 126). As such, one light emitting arrangement may provide light from two light sources which is received as reference light at each of a plurality of optical detectors. The plurality of optical detectors may be distributed about different locations on the subject's scalp (e.g. so as to provide information relating to different regions of the subject's brain tissue). Some of the optical detectors may be clustered together on the scalp (e.g. all located in the same region on their scalp, in close proximity to each other). Where the optical detectors are arranged in close proximity to each other, the controller may be configured to process the combined light signals based on data from a plurality of said detectors (e.g. based on a combination of their sensor outputs). For example, the controller may average the data from at least some of the different detectors to determine properties for the same region of the subject's brain tissue. By comparing data from detectors in roughly the same region of the subject's scalp, the signal to noise ratio may be increased.

Examples described herein relate to arranging the iNIRS system on the subject's scalp, and using the iNIRS system to perform neuroimaging and analysis of a subject's brain. However, the iNIRS system may be used to provide imaging of any suitable subject. As described, the iNIRS system may be used to non-invasively obtain an indication of blood oxygenation levels for the subject. This could be used for any suitable region of their body where blood oxygenation levels are to be sensed, such as in their limbs (e.g. at their wrist, or on their legs). The iNIRS system may be configured to obtain blood oxygenation levels for any suitable portion of the subject's body.

In examples described above, two light sources are used, with a first light source emitting at wavelengths above an oximetry isosbestic wavelength and a second light source emitting at wavelengths below the oximetry isosbestic wavelength. However, this should not be considered limiting. At the two different wavelengths, absorption (and scattering) coefficients for the volume being imaged are likely to be different. This will be due, at least in part, to the differences in absorption characteristics for oxygenated and deoxygenated haemoglobin at different wavelengths. By using known data for these absorption characteristics, a relative contribution to the total absorption coefficient from each of the oxygenated and deoxygenated haemoglobin can be determined. From these relative contributions, a blood oxygenation level may be determined. The two different wavelengths used may be selected to be wavelengths having a discernible difference between oxygenated and deoxygenated absorption coefficients. One example for this is either side of the oximetry isosbestic wavelength, but this is not the only option. The wavelengths could both be on the same side of the oximetry wavelength, as long as there is known data about the differences in absorption characteristics for oxygenated and deoxygenated blood. The controller may use this known data to determine the blood oxygenation level based on the absorption coefficients determined for the volume for the first and second light sources.

Additionally, blood oxygenation level may not be the only piece of information extracted. As will be appreciated, the overall absorption coefficient for a volume will have a number of different contributors (with blood oxygenation being the main one). However, further light sources at different wavelengths may be used to extract information relating to other contributors. For example, additional light sources may use light in different wavelengths selected based on relevant absorption properties for other substances to be sensed. For example, water (e.g. wavelengths above 1000 nm) concentration could be detected. For example, chromophores could be detected, such as Cytochrome C Oxidase. For example, the iNIRS system may comprise a plurality of light sources at different wavelengths, with each wavelength selected to be tailored to a key absorption signature of a substance to be detected. The overall absorption coefficients for the volume may then be decomposed to extract information relating to the individual components to be analysed (e.g. blood, water etc.), such as to identify a relevant contribution from each component to the overall signal (e.g. a relevant concentration of those components).

It will be appreciated in the context of the present disclosure that examples described herein are not intended to be limiting. Instead, examples describe certain potential ways of implementing the claimed technology. For example, the iNIRS system is described with a series of optical cables providing channels and probes for coupling those channels to the subject's scalp. However, it will be appreciated that the probes themselves may be part of the optical channels, or probes may not be provided at all. Similarly, the arrangement of reference channels is just intended to show that reference light is delivered from the light source to the light detectors via optical channels (rather than via the subject's brain tissue). For example, each light source may include one reference channel for each light detector, where that reference channel directly connects the light source to the light detector. In which case, there may be no reference connections in the system at all. Alternatively, and as shown in FIG. 1, the reference light may be transmitted on a common reference optical channel, where some of that reference light is taken from the common reference optical channel to each of the optical detectors. The light source may also be arranged to deliver light to one of a plurality of different locations on the subject's scalp. For example, the light source may be coupled to a plurality of different sample delivery channels, each extended towards the subject's scalp (e.g. from a light splitter).

In examples described herein, there are two light sources, e.g. two separate lasers (one for each different wavelength range). However, in other examples, such as those with time-multiplexing for laser operation, the same light source may provide both the emission of light above and below the isosbestic wavelength. In examples shown in the Figs. a common reference delivery channel 126 is used which may deliver reference light from both the first light source 120a and the second light source 120b towards the one or more detectors. However, this need not be considered limiting, as in other examples, each light source may be coupled to an associated reference channel which couples that light source to a corresponding detector. Each optical detector may be arranged to receive reference light direct from each light source and sample light travelling to the detector from each light source via the subject's brain tissue. The reference light could travel along a common reference channel, or along individual reference channels. Each light source may be coupled to a common reference channel via one or more other channels, such as other reference channels (e.g. as shown in FIG. 2b). In FIG. 2a, a common light combiner 122, a common light channel 123, and a common light splitter 124 are shown, but this division need not be considered limiting. For instance, this functionality may be provided by a light combiner and splitter—e.g. so long as the split light signals onto both sample and reference arms will contain light from either/both light sources.

It will be appreciated that the particular arrangement shown for signal processing circuitry of the detector need not be considered limiting. Each light detecting arrangement 130 is configured to combine sample and reference light to provide a combined light signal with components at one or more beat frequencies, and to process those combined light signals to determine one or more properties of the subject's brain tissue. Any suitable signal processing and/or conversion circuity could be used for this. For example, a transimpedance amplifier may not be needed (e.g. depending on the photodetector/ADC, no current to voltage conversion may be needed, or this may be performed in a different way). Similarly, a balanced photodetector need not be used, and instead a single photodetector, such as a photodiode, could be used. Similarly, the arrangement with the ADC shown in the Figs. need not be considered limiting. For example, multiple ADCs may be used (e.g. one for each detector output stream), or all detector output streams may be fed into one common ADC.

It will be appreciated from the discussion above that the examples shown in the figures are merely exemplary, and include features which may be generalised, removed or replaced as described herein and as set out in the claims. With reference to the drawings in general, it will be appreciated that schematic functional block diagrams are used to indicate functionality of systems and apparatus described herein. In addition the processing functionality may also be provided by devices which are supported by an electronic device. It will be appreciated however that the functionality need not be divided in this way, and should not be taken to imply any particular structure of hardware other than that described and claimed below. The function of one or more of the elements shown in the drawings may be further subdivided, and/or distributed throughout the apparatus of the disclosure. In some examples the function of one or more elements shown in the drawings may be integrated into a single functional unit.

As will be appreciated by the skilled reader in the context of the present disclosure, each of the examples described herein may be implemented in a variety of different ways. Any feature of any aspects of the disclosure may be combined with any of the other aspects of the disclosure. For example method aspects may be combined with apparatus aspects, and features described with reference to the operation of particular elements of apparatus may be provided in methods which do not use those particular types of apparatus. In addition, each of the features of each of the examples is intended to be separable from the features which it is described in combination with, unless it is expressly stated that some other feature is essential to its operation. Each of these separable features may of course be combined with any of the other features of the examples in which it is described, or with any of the other features or combination of features of any of the other examples described herein. Furthermore, equivalents and modifications not described above may also be employed without departing from the invention.

Certain features of the methods described herein may be implemented in hardware, and one or more functions of the apparatus may be implemented in method steps. It will also be appreciated in the context of the present disclosure that the methods described herein need not be performed in the order in which they are described, nor necessarily in the order in which they are depicted in the drawings. Accordingly, aspects of the disclosure which are described with reference to products or apparatus are also intended to be implemented as methods and vice versa. The methods described herein may be implemented in computer programs, or in hardware or in any combination thereof. Computer programs include software, middleware, firmware, and any combination thereof. Such programs may be provided as signals or network messages and may be recorded on computer readable media such as tangible computer readable media which may store the computer programs in non-transitory form. Hardware includes computers, handheld devices, programmable processors, general purpose processors, application specific integrated circuits (ASICs), field programmable gate arrays (FPGAs), and arrays of logic gates.

Any controller of the present disclosure may be implemented with fixed logic such as assemblies of logic gates or programmable logic such as software and/or computer program instructions executed by a processor. The controller may comprise a central processing unit (CPU) and associated memory, connected to a graphics processing unit (GPU) and its associated memory. Other kinds of programmable logic include programmable processors, programmable digital logic (e.g., a field programmable gate array (FPGA), a tensor processing unit (TPU), an erasable programmable read only memory (EPROM), an electrically erasable programmable read only memory (EEPROM), an application specific integrated circuit (ASIC), or any other kind of digital logic, software, code, electronic instructions, flash memory, optical disks, CD-ROMs, DVD ROMs, magnetic or optical cards, other types of machine-readable mediums suitable for storing electronic instructions, or any suitable combination thereof. In particular, any controller of the present disclosure may be provided by an ASIC.

Other examples and variations of the disclosure will be apparent to the skilled addressee in the context of the present disclosure.

1. An interferometric near infrared spectroscopy, iNIRS, system comprising:
a light emitting arrangement comprising:
a first light source configured to provide wavelength-swept emission of light through a plurality of different wavelengths in a first wavelength range;
a second light source configured to provide wavelength-swept emission of light through a plurality of different wavelengths in a second wavelength range different to the first wavelength range;
a common sample delivery channel coupled to each of the first and second light sources to receive light therefrom and to deliver said light towards a subject; and
one or more reference channels for receiving reference light from the first light source and/or the second light source;
a light detecting arrangement comprising an interferometric optical detector;
wherein the optical detector is coupled to the one or more reference channels for receiving reference light from the first and second light sources;
wherein the light detecting arrangement is arranged to be coupled to the subject for the optical detector to receive sample light from the first and second light sources, the sample light comprising light which travelled along the common sample delivery channel;
wherein the optical detector is arranged to combine the sample light with the reference light to provide combined light signals comprising one or more components at a beat frequency between sample light and reference light; and
wherein the iNIRS system is configured to process the combined light signals for providing imaging and analysis of the subject.

2. The iNIRS system of claim 1, wherein the optical detector is configured to: (i) combine sample light from the first light source with reference light from the first light source, and (ii) combine sample light from the second light source with reference light from the second light source.

3. The iNIRS system of claim 2, wherein the iNIRS system comprises a controller configured to determine an indication of scattering and/or absorption coefficients for sample light from the first light source and sample light from the second light source.

4. The iNIRS system of claim 3, wherein the controller is configured to determine an indication of blood oxygen concentration based on the absorption coefficients for sample light from the first and second light sources.

5. The iNIRS system of claim 4, wherein the controller is configured to determine the indication of blood oxygen concentration based on blood absorption data indicating relative absorption coefficients for oxygenated and de-oxygenated haemoglobin at wavelengths of light in the first and second wavelength ranges.

6. The iNIRS system of claim 2, wherein the controller is configured to determine an indication of optical path lengths for sample light from the first and second light sources based on beat frequencies present in their associated combined light signals.

7. The iNIRS system of claim 6, wherein the controller is configured to determine an indication of depth-resolved scattering and/or absorption coefficients for the subject based on determined scattering and absorption coefficients associated with different optical path lengths.

8. The iNIRS system of claim 7, wherein the controller is configured to determine an indication of depth-resolved blood oxygen concentration for the subject based on the determined depth-resolved absorption coefficients.

9. The iNIRS system of claim 8, wherein the controller is configured to determine an indication of a blood flow index for the subject for each of the first and second wavelength ranges.

10. The iNIRS system of claim 9, wherein the controller is configured to determine an indication of a total tissue oxygenation metabolism for the subject based on the determined blood flow index and the blood oxygenation concentration for the subject.

11. The iNIRS system of claim 1 , wherein the first wavelength range includes wavelengths above an oximetry isosbestic wavelength and the second wavelength range includes wavelengths below the oximetry isosbestic wavelength.

12. The iNIRS system of claim 1, wherein the system is configured to be coupled to the subject's scalp for providing neuroimaging and analysis of the subject's brain tissue.

13. The iNIRS system of claim 1, wherein the iNIRS system comprises a plurality of interferometric optical detectors, wherein each detector is coupled to the one or more reference channels for receiving reference light from the first and second light sources, and wherein each optical detector is arranged for receiving sample light from the subject.

14. The iNIRS system of claim 1 , wherein the light emitting arrangement comprises a light combiner arranged to combine light from each of the first and second light sources onto a common optical channel.

15. The iNIRS system of claim 14, wherein the common optical channel is coupled to the common sample delivery channel, or wherein the common optical channel is the common sample delivery channel.

16. (canceled).

17. The iNIRS system of claim 1, wherein the light emitting arrangement comprises:
a first light splitter arranged to split light from the first light source onto a first sample delivery channel and a first reference delivery channel;
a second light splitter arranged to split light from the second light source onto a second sample delivery channel and a second reference delivery channel; and
a sample common light combiner arranged to combine light from the first sample delivery channel and light from the second sample delivery channel onto the common sample delivery channel.

18. The iNIRS system of claim 1 , wherein the light emitting arrangement comprises:
a common light combiner arranged to combine light from the first and second light sources; and
a common light splitter coupled to the common light combiner and arranged to split light received from the common light combiner onto the common sample delivery channel and a common reference channel.

19. (canceled).

20. The iNIRS system of claim 1, wherein the light emitting arrangement comprises a wavelength division multiplexer and the light detecting arrangement comprises a wavelength division demultiplexer;
wherein the light emitting arrangement is configured to provide emission of both light from the first light source and light from the second light source, wherein said emission is wavelength multiplexed; and
wherein the light detecting arrangement is configured to wavelength demultiplex the combined light signals so that combined light signals associated with the first light source are processed separately to combined light signals associated with the second light source.

21. (canceled).

22. (canceled).

23. (canceled).

24. An interferometric near infrared spectroscopy, iNIRS, method comprising:
operating a first light source to provide wavelength-swept emission of light through a plurality of different wavelengths in a first wavelength range;
operating a second light source to provide wavelength-swept emission of light through a plurality of different wavelengths in a second wavelength range different to the first wavelength range;
directing light from each of the first and second light sources on to a common sample delivery channel and towards a subject;
directing reference light from the first and/or second light source onto one or more reference channels;
combining, at an interferometric optical detector, reference light received from the one or more reference channels and sample light received from the subject, the sample light comprising light delivered towards the subject through the common sample delivery channel, to provide combined light signals comprising one or more components at a beat frequency between sample and reference light; and
processing the combined light signals for providing imaging and analysis of the subject.

25. A non-transitory computer program product comprising computer program instructions to program a processor to control operation of an interferometric near infrared spectroscopy system to perform the method of claim 24.
